(19) United States
(12) Patent Application Publication
(10) Pub. No.: US 2009/0074611 A1
(43) Pub. Date: Mar. 19, 2009

(54) PHOTOLYTIC GENERATION OF HYDROGEN PEROXIDE

(75) Inventors: Bruce F. Monzyk, Delaware, OH (US); Richard J. Gilbert, Wellesley, MA (US)

Correspondence Address: Fay Sharpe LLP, 1228 Euclid Avenue, 5th Floor, The Halle Building, Cleveland, OH, 44115-1843, US

(73) Assignee: Battelle Memorial Institute, Columbus, OH (US)

(21) Appl. No.: 12/066,839

(22) PCT Filed: Sep. 15, 2006
(86) PCT No.: PCT/US06/36261

§ 371 Date: Nov. 10, 2008

Related U.S. Application Data
(60) Provisional application No. 60/717,318, filed on Sep. 15, 2005.

(51) Int. Cl. A61L 2/18; B01J 19/00; C01B 15/01

(52) U.S. Cl. 422/29; 422/211; 422/187; 423/584

(57) ABSTRACT

The present invention is directed to a photolytic hydrogen peroxide generator (10); the photolytic hydrogen peroxide generator converts water to activated oxygen for electrolyte absorption, regulates pH, removes hydrogen and other gases; the photolytic hydrogen peroxide generator includes a photolytic cell (16) where chemical reactions occur.

This application claims the benefits of U.S. Provisional Application No. 60/717,318, filed Sep. 15, 2005, and is a continuation in part of U.S. application Ser. No. 10/939,699, filed Sep. 13, 2004; which is a divisional application of U.S. application Ser. No. 09/920,385 now U.S. Pat. No. 6,866,755. The disclosures of Provisional Application 60/717,318 and Nonprovisional application Ser. No. 10/939,699 are incorporated by reference herein.

### FIELD OF THE INVENTION

The present invention is directed to a photolytic cell that utilizes light energy to achieve activated oxygen production (e.g. hydrogen peroxide) as vapor or as an aqueous solution. The invention also includes a method for activated oxygen production. It is to be appreciated, that the invention will also find applications in chemical process industry and the medical fields as in chemical production involving peroxidation, and in disinfection, sterilization or decontamination.

### BACKGROUND OF THE INVENTION

The present technology is a subset of a broader technology platform, termed Photolytically Driven Electro-Chemical technology, or PDEC. This platform brings together several physical systems within close proximity of each other to obtain synergistic interactions. Such a system includes one or more of:

1. A aqueous phase, typically containing one or more optional peroxide stabilizers, and in preferred embodiments an optional pH buffer (typical useful buffers and/or stabilizers include one or more of carbonates, carboxylates, amino acids, pyrophosphates, borates, orthophosphates, Goodes buffers, amino phosphates, colloidal metal oxides such as stannic oxide, and the like).
2. Photolytic energy which provides energy to drive “charge separation” or “exciton” generation whose energy is utilized to drive certain oxidation/reduction and protonation desirable chemical conversions.
3. Electrical energy derived from the charge separation used to optionally drive useful cathodic reactions, whereas electrical energy is derived, at least in part, from the photolytic energy and the exciton electron from the semi-conductor electrical conductance band.
4. Photolytically driven anodic (oxidative) chemical reactions using the “charge separation” energy derived, at least in part, from the photolytic energy and the exciton “hole” from the metal oxide semi-conductor photocatalyst.

Photolytically driven electrochemistry offers a highly controllable means for safely causing major thermodynamic changes, and thus comprises the basis for the biotechnological platform described herein. The present invention uses photolytic generation of H₂O₂ to provider generation on demand or constant generation and a regulated sterilizing chemical environment for the sterilization of various surgical instruments, medical instruments, needles for injection and the like. The invention is useful for disinfecting or sterilizing surfaces, volumes meats, vegetables and wounds in hospitals, ambulances, medical centers, and food processing facilities; instruments, gear, and living quarters for space travel; industrial settings, ambulatory, home use and the like.

Art related to the present application includes:

U.S. Pat. No. 4,094,751 to Nozik, Photochemical Diodes; U.S. Pat. No. 4,889,604 to Kahn et al., Process for the Photocatalytic Decomposition of Water into Hydrogen and Oxygen; U.S. Pat. No. 5,799,912 to Gonzales-Martin et al., Photocatalytic Oxidation of Organics using a Porous Titanium Dioxide Membrane and an Efficient Oxidant; U.S. Pat. No. 6,051,194 to Peill et al., TiO₂ Coated Fiber Optic Cable Reactor; U.S. Pat. No. 6,183,695 to Godec et al., Reagentless Oxydation Reactor and Methods using Same; U.S. Pat. No. 6,866,755 to Monzyk et al., Photolytic Artificial Lung; and WO 01/70396 A2 to Speer, Photolytic and Photocatalytic Reaction Enhancement Device.

### SUMMARY OF THE INVENTION

In one aspect, the present invention is directed to a photolytic cell. It may be utilized for H₂O₂ production for in situ use or generation of peroxide at a remote site, especially as a portable device.

The photolytic cell is a device that utilizes light, such as a laser or lamp or solar, to achieve hydrogen peroxide production.

In another aspect, the photolytic H₂O₂ production cell can be deployed with effects of the UV lamp, ozonizer, ethylene oxide treatment or steam generator, in any combination to impart more extensive disinfection, decontamination or sterilization. These illumination means can be powered from many fuel sources, including H₂ fuel cells (H₂ derived from the photolytic cell), solar powered, and conventional electrical sources.

More particularly, the photolytic cell includes a photoelectrochemical cell (or “photolytic cell”) that, in part, operates similar to the photosynthesis process that takes place in green plants in which a peroxo metal ion oxide cluster produces oxygen gas via a peroxide intermediate In the case of photosynthesis the metal ion cluster is a tetramer of Mn. The invention described herein is not so limited in the metal ions that can be used. The photolytic hydrogen peroxide generator utilizes the photolytic cell and light energy to simultaneously generate hydrogen peroxide from water, useful acidity and electrical energy. One or more photolytic cells can be included in the photolytic cell array of the present invention depending on the quantity, production rate, concentration, etc. of desired peroxide produced. The manner in which such multiple photolytic cells are integrated together is another aspect of the invention.

The light energy utilized in the present invention is any light able to provide sufficient energy to provide photolysis. Typically this is ultraviolet (“UV”) light or visible light 750 nm or shorter, with the UVA and UVB forms being the most preferred. However, the light energy can also be broad-band, received by the way of a “light pipe” fiber optic cable or by the way of an attenuated total reflectance (ATR) design link. Solar energy is also acceptable due to its high power in this wavelength region.

Photolysis is the driving of a chemical reaction as a result of absorbing one or more quanta of radiation. Here, water, hydroxide or oxide ions are converted into activated oxygen which ultimately forms hydrogen peroxide by using a specifically designed light-activated catalyst, such as a semiconducting metal oxide or a blend of such oxides. The metal oxide is utilized as a photo-absorbent material or a photo-absorption element. It is photolytically irradiated to form, from water present in an aqueous solution or provided as vapor or condensation, hydrogen ions, hydrogen peroxide or other forms of oxygen gas precursor (activated oxygen, “AO”), and electrons, by the absorption of one or more quanta of electromagnetic radiation. Critically, the free electrons generated are simultaneously electrically conducted away from the AO to avoid reversal of the reaction to reform water. Optionally the electric power can be utilized to drive electrical devices, such as a pump, and/or to be combined with the hydrogen ions in a subsequent reaction.

For example, it has been found that activated oxygen is readily generated in the present invention by the use of ZnO as the light absorbent photocatalyst material. The metal oxide photocatalyst can be in the form of films, particles, suspended granules, fine powder, porous ceramic, and the like. The photo energy of light, such as ultraviolet laser light (about 350-400 nm), selectively excites ZnO semiconductor transition (about 350-390 nm band, or about 3.1 eV) with minimal material radiation or transmission. The ultraviolet energy produces charge separation in the ZnO referred to as excitons, which then produces activated oxygen (AO) and free electrons. The free electrons are then subsequently electrically conducted away due to the semi-conducting property of the selected metal oxide photocatalyst, for example selected from ZnO, TiO₂, CeO₂, SnO₂, Nb₂O₅, WO₃, and the like, including mixtures of these oxides with or without sensitizing dyes and/or dopant metals and other elements. Alternatively, other suitable light absorbent materials can also be utilized in the present invention at various wavelengths provided that the energy is sufficient to produce activated oxygen.

Disproportionation is a chemical reaction in which a single compound serves as both oxidizing and reducing agent and is thereby converted into a combination of a more oxidized and a more reduced derivative. For example, hydrogen peroxide (activated oxygen) produced during photolysis can be converted by means of manganese dioxide (MnO₂), or other such redox active catalytic agents and/or processes, into dissolved oxygen (DO) and water. This reaction produces dissolved oxygen (DO) and is to be avoided in the production of activated oxygen in order to produce hydrogen peroxide efficiently.

### Photolysis and Charge Separation:

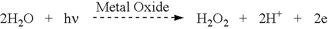

Disproportionation: (to be avoided in the production of H₂O₂)

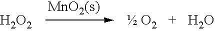

Additionally, the mix of products generated by the photolytic cell of the invention, can be used in to provide point-of-use chemicals such as hydrogen peroxide. The ability to produce electrical power can further be utilized in portable devices and remote locations, for example in powering small pumps, controls, sensors, LED indicators and switches.

In a further aspect, the present invention is also directed to a photolytic cell. The photolytic cell includes a transparent light conduit, light pipe and/or window. An electrical conductor is adjacent to the transparent window. A light-activated catalyst abuts the electrical conductor. A cell flow through space is adjacent to the light activated catalyst. Optionally, a cation exchange membrane borders the cell flow through compartment. A catholyte compartment abuts the cation exchange membrane, if present, and a cathode. A cathode is present adjacent to the catholyte and is electrically connected to the anode either directly or via an in-line electrical device. The cathode receives electrons via the electrical conductor at the photo anode.

These and other objects and features of the invention will be apparent from the detailed description set forth below.

### BRIEF DESCRIPTION OF THE DRAWINGS

The present invention will become more fully understood from the detailed description given below and the accompanying drawings. The description and drawings are given by way of illustration only, and thus do not limit the present invention.

FIG. 1A shows a perspective view of an embodiment of a typical portable hydrogen peroxide generator designed for portable use.

FIG. 1B is a schematic diagram of a broad aspect of the invention showing one photoelectrochemical cell.

FIG. 1C is a schematic diagram of one embodiment of the invention showing a general illustration of one cell of the photolytic hydrogen peroxide generator connected externally to a treatment volume, tank, or chamber.

FIG. 1D is a schematic diagram of one embodiment of the invention showing a general illustration of one cell of the photolytic hydrogen peroxide generator with a recirculation loop.

FIGS. 2A-2F illustrate the various embodiments of the photolytic hydrogen peroxide generator set forth in FIGS. 1A and 1B.

FIG. 2A shows an interior view of one cell of one embodiment of the hydrogen peroxide generator wherein light enters on flat side of a waveguide.

FIG. 2B shows an interior view of the components of one cell of another embodiment of the photolytic hydrogen peroxide generator wherein the light enters on the end of a waveguide.

FIG. 2C also shows an inside view of an alternative embodiment of the photolytic hydrogen peroxide generator wherein a protective layer 220 is coated on the active layer 215.

FIG. 2D illustrates a cell configuration where the active layer is a dual active layer. including a first active layer and a second active layer.

FIG. 2E illustrates schematically an interior view of an array of cells.

FIG. 2F illustrates schematically an interior view of an alternative embodiment of the hydrogen peroxide generator.

FIG. 3 shows a schematic view of the photolytic cell which was used to collect the laboratory data set forth herein.

FIG. 4 shows an overall schematic diagram of one preferred embodiment of the photolytic hydrogen peroxide generator of the present invention.

FIG. 5 is a schematic drawing of another embodiment of an apparatus for sterilization including a cell for producing hydrogen peroxide.

FIG. 6 is a schematic drawing of another embodiment according to the invention illustrating application of an optional bias voltage to a photochemical cell.

FIG. 7 shows a graph illustrating the relationship of the pH profile of the anolyte and catholyte during photolysis using the photolysis cell.

### DETAILED DESCRIPTION AND BEST MODE

Broadly, the present invention is directed to a photolytic hydrogen peroxide generator having, among other components, a photolytic cell. The photolytic cell is the fundamental functional unit of the invention. It acts as a general purpose activated oxygen producer. The photolytic cell includes a photochemically active material for use in converting water (H₂O) into activated oxygen (normally peroxide ion (O₂²⁻), which then forms aqueous hydrogen peroxide (H₂O₂) or its conjugate base (HO₂⁻). By optimizing a relative energy band gap balance between photocatalyst, photolytic cell surface, H₂O in liquid or vapor form, and electron removal, it is designed to maximize efficient H₂O₂ generation.

In the preferred embodiment, the present invention is directed to the use of the photolytic cell in a decontamination device and process, i.e., a photolytically driven hydrogen peroxide generator. The photolytic hydrogen peroxide generator includes one or photolytic cells having photochemically active material and associated components for the production of activated oxygen for hydrogen peroxide generation and/or hydrogen peroxide directly, including its salts. Optionally, the invention may include a photolytic chamber to house or hold a sufficient number of stacked or assembled photolytic cells to perform the rate of gas exchange desired. The number of stacks is such to provide sufficient anode surface area for the application and could be micro-cell sized or could be fabricated into medium or much larger areas.

Preferably, the photolytic hydrogen peroxide generator of the present invention comprises water, an electrolyte or water vapor inlet; a pump(s) or allowance for gravity flow, in some embodiments a filter, at least one photolytic cell, a light source(s) that irradiate the photolytic cells, and optionally, a hydrogen gas separator. A power source and/or batteries can be present to power the pump(s) or light source(s). One or more in-line sensors, for example ORP (oxidation-reduction potential) infrared, conductivity, oxygen and/or sensors, and electronic controllers/processors can be present to monitor and optimize the flow through the system, the amount of active peroxygen production, the presence of chemicals, toxins, pollutants, peroxide stabilizers/destabilizers, etc. Aqueous solution circulating through the device will be pumped or gravity fed through the photolytic cells where light activation will result in peroxide generation and hydrogen gas co-production, or other reduced co-product. Catholyte and anolyte electrolyte flows are contemplated to be controlled to be the same or different in the presence of a divider that is a membrane. In a preferred embodiment, the membrane is not present and the H₂ gas is quickly separated from the anolyte. In the most preferred embodiment, the membrane is replaced by a screen of fine opening size.

An alternate embodiment of the invention provides for vaporization or aerosolization of the produced hydrogen peroxide. The vaporized hydrogen peroxide normally, but not necessarily, co-mixed with water vapor and/or a noncondensable carrier gas, such as air, can then be used in a sterilization chamber or otherwise administered as a vapor, gas, fog, or mist to a surface or volume. Vaporization may be by vacuum and/or thermally driven flash evaporation or other known methods. Typically, flash evaporation is with a heating element or steam jacket. The heating element and/or evacuated attachment may be placed between the outlet of the hydrogen peroxide generator and the treatment volume discussed further herein.

A further embodiment of the invention provides for a hand-held hydrogen peroxide generator that can be used as a portable generator for treating selected surfaces or volumes with a hydrogen peroxide containing vapor, fog or mist.

In a yet further embodiment hydrogen peroxide can be reacted under appropriate alkaline (pH>7) pH to form reactive HO₂⁻ that is also useful for more aggressive disinfection or sterilization. Hydrogen peroxide can be reacted to form useful peracids and their salts. Thus, carboxylic acids can be reacted from percarboxylic acids that are also useful for disinfection or sterilization. Borates, phosphates, sulfates (preferably as esters), and the like can likewise be reacted.

Also, the present photolytic portable generator does not require the careful control of temperature or pressure. As briefly mentioned above, substantially all materials for use in the present photolytic hydrogen peroxide generator remain as insoluble solids to prevent loss of materials and solution contamination. Diffusion layers, and/or electron/hole recombination reactions, which can dramatically decrease activated oxygen production rates, are minimized by not incorporating gaseous dissolution, multiple membranes, large internal volumes, or multiple treatment steps, and by using electrical conduction removal of electrons and cations from the photolytic site, and high concentration of H₂O at the activated oxygen formation site, as is done in photosynthesis, by incorporating thin films, having good photolytic transparency, and good electrical conduction and fast electrochemical reactions.

The wavelength, beam size, pulse duration, frequency, and photon flux intensity of the light source are adjusted to produce maximum and/or efficient activated oxygen e.g., hydrogen peroxide generation. Similarly, pump rate, flow-through capacity, etc. of the photolytic cells are also adjusted with activated oxygen concentration in the anolyte exiting the cell being indirectly proportional to the anolyte flow rate when all other conditions are fixed. This flow control is accomplished by sensors and regulators that also monitor reaction chemistry, toxins, etc. The sensors and regulators have the capacity to auto-regulate various parameters of the system in response to the conditions monitored by the sensors.

Most preferably, the activated oxygen produced by the invention is hydrogen peroxide. In one example, for medical device disinfection, the photolytic hydrogen peroxide generator is designed to provide at least 150 ml of dissolved H₂O₂ per minute at 5 L/min of sterilizing solution flow through the system for a treatment volume. Also, the components utilized for construction of the photoactivated disinfection device are essentially nonreactive with the aqueous electrolyte solution or the activated oxygen, normally hydrogen peroxide.

The photolytic hydrogen peroxide generator can be designed so that it is a permanent installation or a portable device.

H₂O₂ is an excellent sterilization reagent because it is an effective biocide, environmentally neutral and does not form hazardous products. However, H₂O₂ is known to readily decompose via disproportion, to oxygen gas, water, and heat. While this instability is useful after it's use in that it guarantees the lack of residual oxidant upon discharge, this is also problematic in that the premature decomposition of H₂O₂ compromises its very purpose of disinfection. In order to counter this problem, one may employ mechanisms by which disproportionation is minimized and/or by which H₂O₂ supply or production is maintained or both. Although pure H₂O₂ is quite stable if stored and handled properly by experts in a few specially fabricated large facilities, this is difficult to achieve practically for the thousands of end-use locations where it is needed in small quantities. This situation arises from the fact that, in use, H₂O₂ can be exposed to a variety of conditions, which enhances its rate of decomposition, which occurs rapidly, in fact usually within seconds. What is more, monitoring H₂O₂ strength in process solutions is difficult to perform routinely. There are generally believed to be five decomposition pathways for H₂O₂, all of which are autocatalytic or are known to feed into autocatalytic processes. Autocatalytic chemical reactions are those, once initiated, that produce their own intermediates for continued reaction. These pathways include thermal decomposition, catalytic decomposition, heterogeneous catalysis of H₂O₂. disproportionation. oxidation of metal, and alkaline destabilization. These are to be avoided to the extent possible.

To limit the problem of hydrogen peroxide decomposition, inert materials are preferable for construction of devices to produce and hold peroxide compounds and solutions (e.g. aluminum, pure plastics such as PVDF, Teflon, polyethylene, and the like) and production processes target high purity process streams. H₂O₂ stabilizers have also been useful where such stabilizers do not interfere with the use of the H₂O₂. Added stabilizers generally target the blocking of one or several decomposition mechanisms, especially providing sequestration of dissolved metal ions capable of catalyzing the autocatalytic decomposition of peroxides. For the invention, certain chelants, such as the oxidatively resistant chelating phosphonates, aminophosphates, amino carboxylates and especially pyrophosphates, are used to bind metal ions tightly to prevent their fast redox cycling reactivity by thermodynamically stabilizing the higher oxidation state as a chelate complex, thus rendering them substantially less catalytic. Stannates, borates and inorganic phosphate colloids are also useful for encapsulating these metal ion decomposition catalysts within colloids and/or precipitates. As an additional example, organic free radical traps, such as acetanilide, prevent peroxide retard decomposition by maintaining a low population of free radical intermediates key to maintaining chain reactions, by free-radical scavenging reactions, thus reducing the rate and likelihood of initiation and continuance of autocatalytic reactions. We note however, that stabilizers only slow the decomposition of peroxides as long as solutions of the peroxides are maintained pure with respect to the particulates, metal ions and other decomposition catalysts. Once the peroxide solution is contaminated by external material, for example when soiled medical surgical tools are submerged in the bath for sterilization, then peroxide strength/concentration can become weak rapidly due to autocatalytic disproportionation and oxidative losses requiring regular peroxide replenishment.

Broadly, according to one aspect of the invention, hydrogen peroxide is produced in useful quantities for sterilization and other uses by photolytic generation from water, in either liquid or vapor form, at a suitable photocatalyst. The metal oxide photocatalysts pure and combinations disclosed herein provide the ability to convert light energy to produce charge separation, excitons, which at selected surfaces can be used to result in the generation of H₂O₂. In this process the production of O₂ is preferably minimized and the production of activated oxygen such as hydrogen peroxide is maximized through choice of catalyst composition and enhanced using specific layering of such catalysts into constructs of thin films.

In another embodiment of the invention, the H₂O₂ so produced photolytically is further concentrated by evaporation and/or distillation in non-catalytic vessels, for example made of aluminum or pure plastics. It can also be pH adjusted upwards to greater than a pH of 9 to enhance its oxidation and disaffecting aggressiveness, or adjusted to a acid or neutral pH for use at milder and more stable conditions. The product peroxide can also be concentrated or vaporized as further means to impart additional oxidation and sterilization or disaffection performance.

The present invention utilizes a semi-conducting metal oxide material photolytic film, as the photo-absorption element and this same oxide semiconductor film, or a blend with one or more cover film of other oxide film or films, exposed to water and/or water vapor for which at least a portion of the H₂O is converted to liquid, solution or vapor H₂O₂, with concomitant release of electrons and hydrogen ions (Reaction 1). For Reaction 1, other bonds to M are not shown for readability but are well known to those skilled in the art and consist of other ions in the oxide particle/film and/or to water or other liquids in the solid/water material representing the invention.

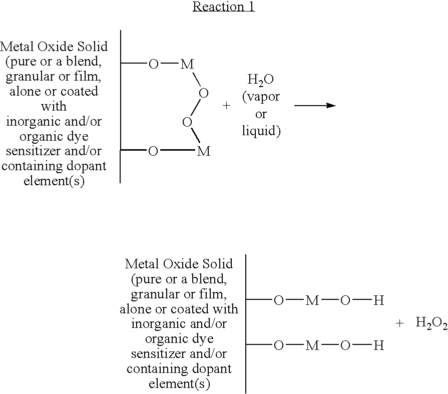

Candidate metal oxide species for activated oxygen generation (activated oxygen being defined as the reactive forms of oxidized oxygen other than O₂ in the ground electronic state) includes a single semiconducting metal oxide (SCMO) photocatalyst, a blend of two or more metal oxide semiconductor photocatalysts, (SCMₓO), where “x” represents blend of differing M components of metal oxide semiconductor metal ions, preferably Zn, Ti, W, Sn, and the like. In addition to the photocatalytic activity of activated oxygen formation, the granular and/or film material of the invention also necessarily contains components capable of H₂O₂ formation via Reaction 1. This material can be one and the same as SCMO or SCMₓO, or another one or materials either blended with SCMO or SCMₓO, or is provided as a full or partial film or coating of H₂O₂ forming metal oxide material over the SCMO or SCMₓO material(s). Such H₂O₂ forming materials are listed in Table 1 and are related by their pKₕ values and/or by their pH values for 50% hydrolysis of the corresponding aquated cation (M(OH₂)_{y}ⁿ⁺), metal ion, Reaction 2.

To produce H₂O₂, the semi-conductor material is typically illuminated by light in the 190 to 750 nm window, but preferably using a wavelength width matched to the performance of the photocatalyst, normally all or part of the range of 340 to 750 nm bandwidth, and most preferably 350-400 nm, thus avoiding wasted energy by transmission or heat generation, and avoiding photo-dissociation of H₂O₂ at wavelengths at less than about 340 nm.

Based on these principles, means to enhance design of the nano and micro-scale architecture around the point of photon sorption and charge separation results in an increased rate of H₂O₂ generation per unit area of photocatalyst and in quantum efficiency. Typical semiconductor materials useful with the invention include those listed in Table 1 and in the discussion above and below used either singly or in combination. These materials are preferably used as illuminated films, where illumination can be accomplished edge on, or from either side, but also are effective in granular and/or powder forms suspended in solution or packed into columns or beds. In certain cases, control of pH of the water phase is most preferred in some embodiments to avoid catalyst dissolutions, such as when ZnO is used. ZnO dissolves under low and high pH or when complexing agents are present. However, when the pH is controlled in range of about 8.5 to 11.5, ZnO has a very low solubility and can be used directly. The use of pH control to limit the solubility of metal ions in aqueous solutions is well known by those in the art and this science is incorporated in this text by reference (For example J. Kragten in “Atlas of Metal-Ligand Equilibria in Aqueous Solution”, Ellis Horwood Limited, 1978 (New York, N.Y.))

TABLE 1
Catalyst Materials For H₂O₂ Production

| | | | Oxidation |
|---|---|---|---|
| Typical Metal | | pH of Initial | States |
| used for Oxide | pKₕ (M⁺ⁿ)⁽²⁾ | Hydrolysis⁽¹⁾ | Common |
| Cadmium (Cd) | 10.1 | 4.6 | |
| Yttrium (Y) | 7.7 | 6.4 | 3 |
| Ytterbium (Yb) | 7.7 | 5.8 | 2⁽³⁾, 3 |
| Terbium (Tb) | — | 6.0 | 3 |
| Samarium (Sm) | 7.9 | 6.1 | 2⁽³⁾, 3 |
| Scandium (Sc) | 4.4 | 4.2 | 3 |
| Nickel (Ni) | 9.8 | 5.7 | 2, 3⁽³⁾ |
| Zinc (Zn) | 8.9 | 6.0 | 2 |
| Lanthanum (La) | 8.5 | 6.5 | 3 |

Comparison Comp.

| | | | |
|---|---|---|---|
| Gallium (Ga) | 2.6 | 1.5 | 2⁽³⁾, 3 |
| Tin (Sn) | 3.4 | 1.2 | 2⁽³⁾, 4 |

⁽¹⁾For a given initial total metal cation concentration the pH is given at which 1% of the total metal ion concentration will start hydrolysis useful for predicting the capability for the production of H₂O₂.
⁽²⁾The pKₕ (M⁺ⁿ) is that pH where 50% of the total metal ion concentration has precipitated as M(OH)⁰ₙ.
⁽³⁾Indicates ion can form unstable oxidation states as marked. Another consideration is that the +2 ions are typically more soluble in aqueous solutions than the +3 ions, or have a narrower pH window of insolubility (e.g. zinc).

In addition to the transitional metals listed in Table 1, the rare earth metals outlined herein are useful with one or more embodiments of the invention.

Therefore there are several requirements for a successful photolytic cell. As a preference, a first set of typical metals useful with the invention preferably have only one essentially stable oxidation state. Metals with essentially only one stable oxidation state will not substantially decompose the hydrogen peroxide that is produced. Examples for these metals include Pr, Nd, Pm, Sm, Gd, Tb, Dy, Ho, Eu, Tm, Yb, Lu, Sc, Y, La, Zn, Cd, In, Al and the like, including combinations thereof. While elements such as Sm, Eu, Sn, and Yb have a second oxidation state (Table 1), the second oxidation state is not stable or is very unstable. For example the +2 state in Eu⁺² is converted to Eu⁺³ in the presence of weak oxidants including air. Thus when hydrogen peroxide is produced in a solution of this metal ion in its +2 oxidation state, the metal ion will be oxidized to +3 (or +4 state in the case of Sn), and remain there, and thus is not able to substantially decompose the hydrogen peroxide by a redox cycling chain reaction mechanism. However, the most preferred list of typical metals used for oxides does not use Sm, Eu, Yb, or Ni. The reason for this is that ions that only have one oxidation state available to them are readily available and these will not result in even small amounts of H₂O₂ losses by the redox cycling mechanism pathway, and thus offer the maximum capability for the highest rates of H₂O₂ production and retention.

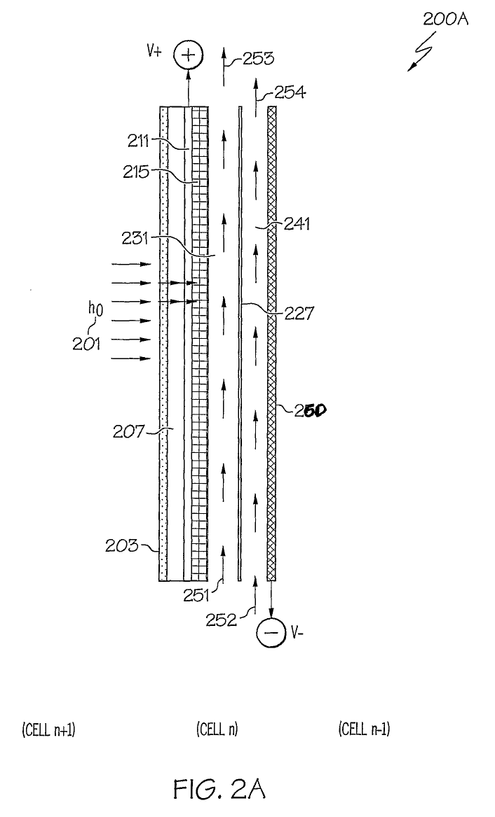

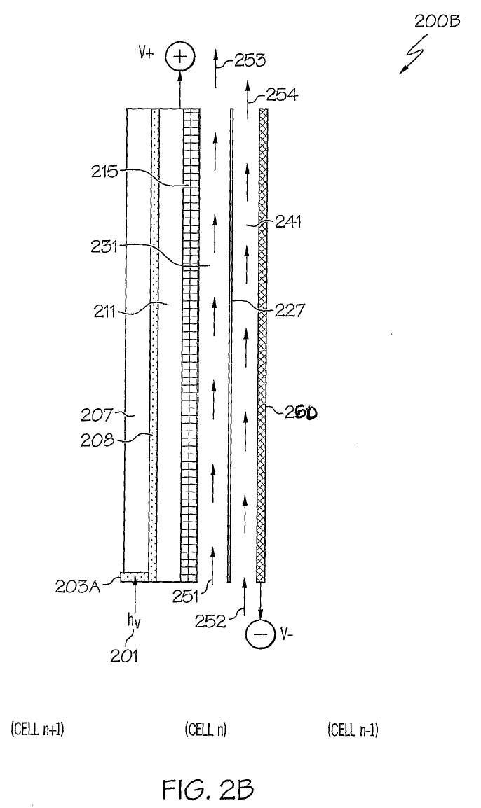

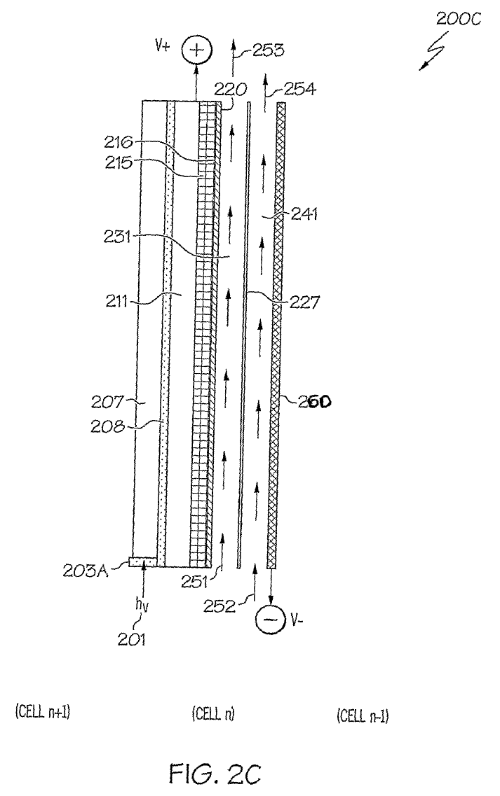

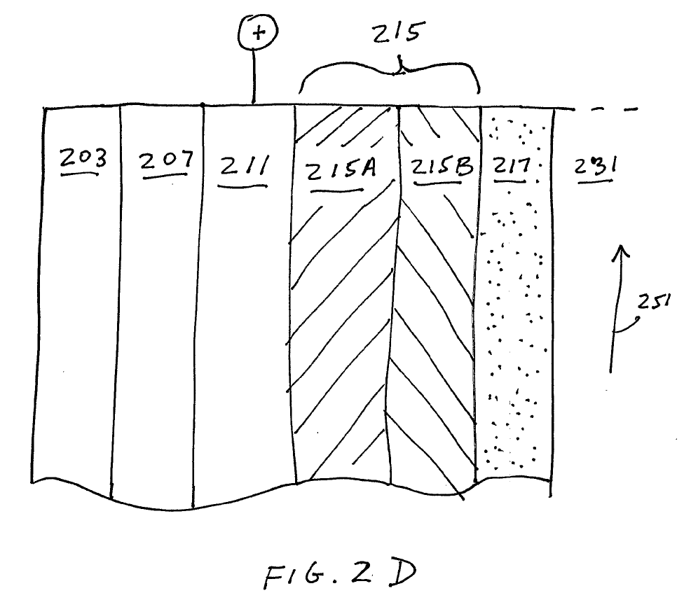

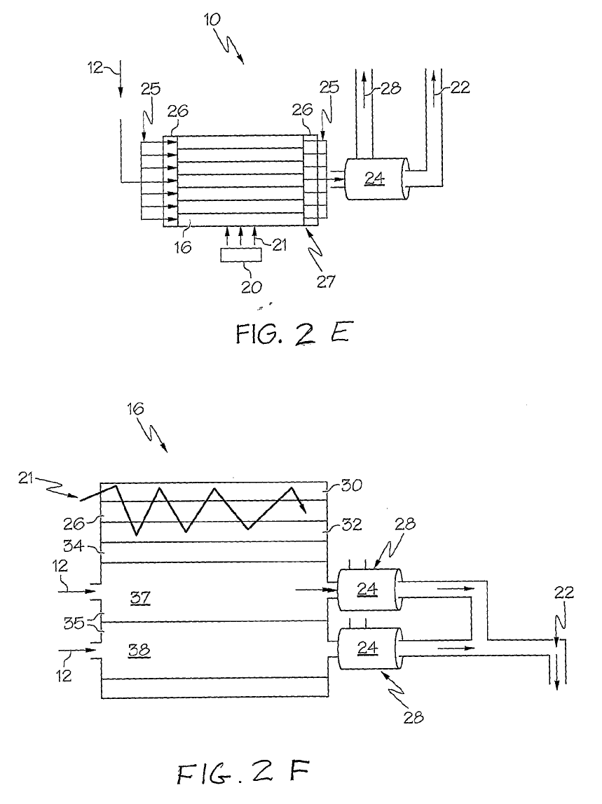

Most preferred is a H₂O₂ generative metal oxide film, coating, or layer (Table 1 component, alone or in combination with others from Table 1) is typically prepared as a photocatalyst metal oxide layer (Table 2 component alone or in combination) oriented such that illumination by a lamp, laser, or solar energy is possible and that produces the activated oxygen followed by hydrogen peroxide production device (see FIGS. 2A to 2D).

Stabilizers added to the water phase for maintaining the optional H₂O₂ product solutions produced by the invention typically include stannic oxide colloid, oxidatively resistant phosphonates, including Dequest® 2010 in its acid form and the like, anilide, acetanilide, isopropylhydroxylamine, BHT, and pyrophosphates such as sodium pyrophosphate. The electrolyte may contain some of metal oxides such as stannic oxide colloids; however, they do not produce hydrogen peroxide; instead they encapsulate the metal ions in solution that would disproportionate hydrogen peroxide if not so encapsulated (e.g. Fe, Cu, Ag, Mn, and the like).

Table 2 shows a list of photocatalyst materials of the invention suitable for charge separation and/or activated oxygen production, especially H₂O₂ production. Preferably these materials are used as illuminated thin coatings (films), but also can be used in granular and/or porous frit, porous pot, or other porous but insoluble forms, where upon illumination is also provided from any functional direction(s), including through the aqueous film or sufficiently transparent solution in contact with the photocatalyst as further discussed herein. As such solutions are easily made clear and colorless, through-solution illumination is one preferred embodiment of the invention so long as the wavelength of light used is greater than about 325 nm to minimize photo-dissociation of the product H₂O₂ or its anions and metal ion salts.

TABLE 2
Photocatalyst Materials Suitable for Generation of Charge Separation
and/or Activated Oxygen Production Upon Illumination

| SEMICONDUCTOR NAME | CHEMICAL FORMULA |
|---|---|
| Titanium Dioxide (anatase) | TiO₂ |
| Titanium Dioxide (rutile) | TiO₂ |
| Titanium Dioxide (anatase/rutile blend) | TiO₂ |
| Tungsten Oxide | WO₃ |
| Zinc Oxide | ZnO |
| Zirconia | ZrO₂ |
| Iron(IV) oxide | FeO₂ |
| Reduced iron oxide | Fe₂O |
| Bismuth oxide | Bi₂O₃ |
| Stannic oxide | SnO₂ |
| Lead(IV) oxide | PbO₂ |
| Strontium Titanate | SrTiO₃ |
| Barium Titanate | BaTiO₃ |
| Ferrous Titanate | FeTiO₃ |
| Potassium Titanate | KTiO₃ |
| Manganese Titanate | MnTiO₃ |

Some embodiments use the herein disclosed metal oxides, the materials of Table 2, which are capable of e⁻/h⁺ charge separation, and so are useful as photocatalysts (#215A of FIG. 2D), but are not used as the second component for hydrogen peroxide production (#215B of FIG. 2D). i.e. by using one or more of TiO₂, ZrO₂, WO₃, FeO₂, Fe₂O, Bi₂O₃, SnO₂, PbO₂, SrTiO₃, BaTiO₃, FeTiO₃, KTiO₃, MnTiO₃, and combinations thereof, for films or coatings for hydrogen peroxide production, since their water hydrolysis constant values (pKₕ, Table 1) are too low. Such materials possess Reaction 1 equilibria that lie to the left, and which therefore tend to release too little H₂O₂, and instead tend to form O₂.

A second property typically useful for the H₂O₂ forming metal oxides herein is that the metal oxides have a pH of first hydrolysis about at or above about pH 4 so that the activated oxygen species, approximated by M-O—O-M, is sufficiently a weak base to allow water to hydrolyze the M—O bonds (Reaction 1). A pH of about 4 to about 13 is preferred to achieve H₂O₂ by production by hydrolysis of M-O—O-M, with a pH of about 4 to about 10 being most preferred. This pH is measured as the pH at 1% hydrolysis of the total metal ion concentration present in an aqueous solution of the metal ion being considered. It is believed that this pH allows hydrolysis of the metal peroxide to hydrogen peroxide (Reaction 1).

A third property typically useful for the photocatalyst metal oxides is that the metal oxide have a charge transfer electron transition in the about 190 nm to about the about 750 nm wavelength range. Organic dye sensitizers and/or metal ion and/or representative element dopants may be used to accomplish the full range of use of the UV and/or visible spectrum up to about 750 nm using photocatalyst with bandgap or higher energies also in the 190-750 nm range. Typical examples of organic dye sensitizers such as dye N-749 black dye, or N-719 dye, Ru bipyridine complexes (described by S. Altobello, et. al. of J. Am. Chem. Soc. 2005, 127, 15342-15343), and the like, bound to the photocatalyst surface using linear poly(ethyleneimine), poly(acrylic acid), polyethylene oxide, and the like. With such refinements quantum yields can typically reach 1 to 10%. Typical examples of dopants include dopants derived from low levels, normally 10 wt % of transition metal ions, lanthanide ions, alkali and alkali-earth ions, organic dyes as compiled by the Chemical Index (C.I.), and representative metal ions and/or representative elements individually or in combination (Se, As, P, S, N), including ions derived from the previous list of elements having only one stable oxidation state. Combinations of these dopants are within the scope of the invention.

A fourth property typically useful for the metal oxide is that, for the cases where liquid water is used to collect the H₂O₂ and to supply H₂O to the H₂O₂ generating surface as liquid or vapor, the solubility in the electrolyte be such that the catalyst film, as is or with protective coating, does not appreciably dissolve in the aqueous solution or electrolyte. Thus the solubility of the metal oxide in the electrolyte is preferably below about 10⁻³ molar (M), more preferably below about 10⁻⁴ M, and most preferably less than about 10⁻⁵ M. For example ZnO has a solubility of about less than 10⁻⁴ Molar. Solubility above or near that of zinc oxide will typically require an additional sealing layer and/or, preferably, electrolyte composition control so as to prevent loss of the H₂O₂ generating active layer. Suitable sealing materials are 1 nm to 10 microns thick porous films of silica, gold, platinum group metals, graphitic carbon, nickel, barium, lead, tin, aluminum metals, blends of these materials, clay-like materials, alumno-silicates, glasses, gels, and the like. Such materials can be prepared by well known vacuum electroless metal plating or sol-gel coating deposition techniques. Silica can also be applied by dipping the photocatalyst/H₂O₂ production construct in sodium silicate at 90° C.-105° C. solution. Low solubility of the H₂O₂ forming surface in water is desired so that the metal oxide layer is not stripped off during H₂O₂ production.

This further typical embodiment of the invention provides for a stabilizing film or sealer on the metal oxide layer that produces the hydrogen peroxide. This is particularly useful where the metal oxide is too soluble in the electrolyte at a given condition (e.g. pH.). The stabilized film is maintained so that the solubility product of the protective film is less than the solubility constant of the metal oxide film to be protected (e.g. ZnO) over a broader pH range (see just above for examples of such materials).

The following examples are intended to be exemplary of the invention and are not intended to limit the invention in any way.

### EXAMPLE 1

Thus a metal oxide film or a particular coating is optionally surface treated to render the film or coating less soluble in electrolyte or water. For example, ZnO or another like metal oxide could be treated with orthophosphate before use or an organic polymer, such as one or a combination of those listed above.

### EXAMPLE 2

Alternatively to Example 1, continuous treatment during use may be achieved by circulating a metal oxide stabilizer in the electrolyte in an amount effective to shield the film or coating against dissolution. Example 2 examples include carbonate, oxalate, ferrocyanide, oxinates, orthophosphates, 8-hydroxyquinoline (quinolinate), molybdates, sulfides, arsenates, molybdates, nitrides, carbides, and pyrophosphates. At least an amount effective to form a protective film is needed wherein the film reduces or substantially eliminates removal of the active oxide if exposed to the corresponding electrolyte of the invention provided as a bulk liquid, as a liquid film, or in vapor or vapor condensate form.

Insoluble sealer(s) consisting of one or more of the following is useful for protecting the active layer: silica, silicate, molybdate, arsenate, chromate(III), aluminate, borate, zirconate(IV), titanate (IV), germanate, cerate and the like.

### EXAMPLE 3

The operational pH of the liquid aqueous film adjacent to the H₂O₂ generating material is typically at or above a pH of about 4. Most preferred a pH range of about 4 to about 13 is useful with the invention. In some embodiments the upper pH limit is that at which 99% of the metal ion has precipitated as M(OH)⁰ₙ or is in M(OH)⁰ₙ colloid form, wherein “n” is determined by the oxidation state of the metal and is either 2, 3 or 4 and where 2-OH can be replaced by one O⁻².

### EXAMPLE 4

Surfaces in contact with the hydrogen peroxide such as containers, piping, storage, pumps, valves and the like are typically made of aluminum metal, or plastics such as polyolefins of the type polyethylene, polypropylene, fluorocarbons and the like that do not easily catalyze with or disproportionate hydrogen peroxide. Purified materials are most preferred but optional since impurities can be flushed away with ease.

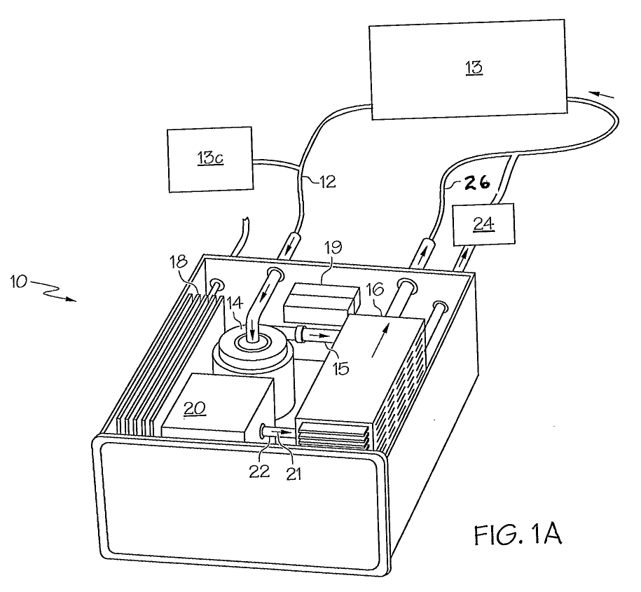

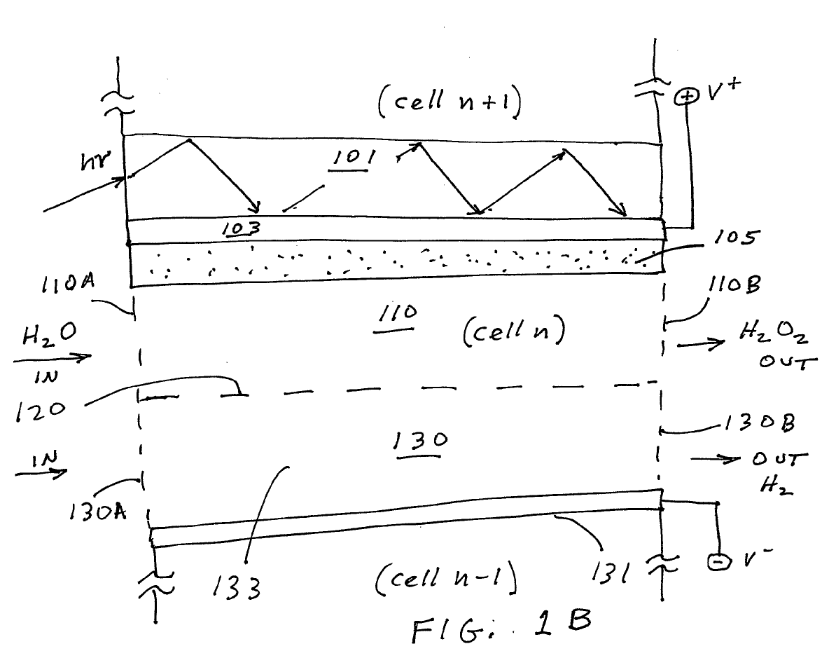

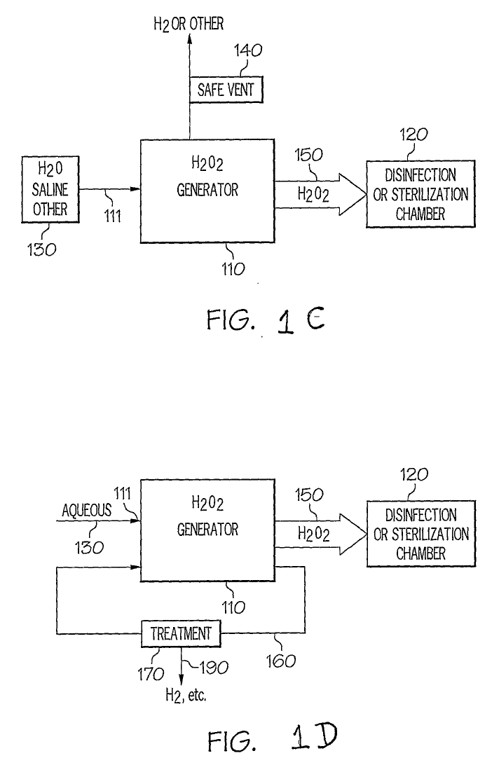

More particularly, FIG. 1 shows an embodiment of a portable hydrogen peroxide generator 10 that provides activated oxygen by a photolytic process. The portable generator 10 includes an aqueous solution inlet 12 that provides for liquid flow from a treatment tank 13 to the portable generator 10. The aqueous solution inlet 12 is connected to an optional pump 14 that draws aqueous solution from the treatment tank or clean solution source 13c (pump is optional) into the portable hydrogen peroxide generator 10. The pump 14 directs aqueous solution through an optional filter 15 or line to one or more photolytic cells 16 where light activation (for example, laser or UVA at 350 to 400 nm) results in activated oxygen generation and ultimate hydrogen gas removal via a gas sorption device 24 or external ventilation. A power supply 18 or optional battery 19 activates the light source 20. The light source 20 emits light photons 21 which irradiate the photolytic cells 16 via a light pipe 22. In turn, the photolytic cells 16 photochemically initiate a series of chemical reactions that produce activated oxygen. Aqueous solution containing activated oxygen travels from the portable generator 10 back to the treatment volume by way of outlet 26

FIG. 1B is a schematic diagram of one cell in a general embodiment of FIG. 1A and illustrates a cell n 100, Additional cells n+1, and n−1 are possible. The cell includes a waveguide 101 for conducting light to and through an adjacent first conductor 103 which is typically an anode. Adjacent to the conductor 103 is an active layer, that may consist of one or more active layers 103 and an optional protective layer (not shown in this view). The active layer is typically bounded by an anodic compartment 110 (first volume or chamber) where hydrogen peroxide is formed at the interface of the active layer and an electrolyte 107 that is within the anodic compartment 110. An optional divider 120 typically forms one portion of the anodic compartment that separates it from the cathode compartment 130 (second volume or chamber). The cathode compartment 130 is typically also bounded by a second conductor 131 which is typically a cathode. The anode compartment typically has an inlet 110A and an outlet 110B. The cathode compartment also typically has an inlet 130A and an outlet 130B. An electrolyte 133 is also typically present in the cathode compartment.

FIG. 1C shows a simplified representation of a system having a treatment volume 120 attached to a photolytic hydrogen peroxide generator 110. Water, saline or other source 130 flows via inlet 111 to the hydrogen peroxide generator 110. Hydrogen peroxide 150 produced in the generator 110 flows to a disinfection or sterilization chamber 120. Hydrogen or other offgases typically flow out via a safe vent 140.

FIG. 1D shows a simplified representation of a system having a treatment volume 120 attached to a photolytic hydrogen peroxide generator having a recirculating loop 160 for the cathodic flow of aqueous solution. Aqueous input 30 flows via inlet 111 to hydrogen peroxide generator 110. Hydrogen peroxide 150 produced in the generator 110 flows to a disinfection or sterilization chamber 120. Recirculating loop 160 provides for cathodic flow to a treatment volume 170. Hydrogen or other gases can be vented at outlet 190.

FIGS. 2A through 2D are enlargement views showing the components of various embodiments of the photolytic hydrogen peroxide generator 110. One embodiment of an apparatus 200A for the production of hydrogen peroxide and its administration to a surface or material to be treated is shown in FIG. 2A. One or more cells are shown in FIG. 2A as cell n, cell n−1, and cell n+1, and so on. A light source not shown (e.g., laser, UVA lamp, or sunlight) provides light hv 201 to an optional antireflective layer 203 or directly to an adjacent window (face-on illumination) or a waveguide 207 (angular or end-on illumination). Waveguide 207 conducts the light 201 through a first optically transparent but electrically conductive layer 211 that is typically biased with a positive voltage by means of one or more of the following: an externally applied potential, a P/N junction, a diode, connected to a thermodynamically favorable cathode chemical reaction, and the like. The light passes from the waveguide 207 through the adjacent first conductor layer 211 that is slotted, has grids, and/or is light transmissive so as to allow the light to reach a catalytic active layer that is located on the opposite side of and adjacent to the first conductor 211. Waveguide 207 is in intimate electrical contact with the catalytic active layer 215. The catalytic active layer 215 must be one that is capable of charge separation upon illumination. The catalytic active layer 215 forms at least in part one boundary of a first volume or chamber 219 (hereinafter first volume). An optional divider 227 separates the first volume 231 from a second volume or chamber 241 (hereinafter second volume). The second volume 241 is bounded in part by the divider 227 and at least in part by a second conductive layer 260 that is typically a cathode. Second conductor layer 260 is typically biased with a negative voltage. During operation, aqueous fluid 251 flows into volume 231 as inflow-1 and aqueous fluid 252 flows into volume 241 as inflow-2. In some embodiments inflow-1 251 may be the same as inflow-2 252. In further embodiments, inflow-1 251 is typically water and an aqueous pH or electrolyte buffer (e.g., orthophosphate salt at pH of about 6-13 for TiO₂ and about 6.5-7.5 for ZnO). Outflow-1 253 typically also carries hydrogen peroxide that has been generated and is typically guided to a disinfection or sterilization site. Inflow-2 252 is typically an aqueous solution consisting of an electrolyte, saline, easily electrochemically reduced reagent and/or other optional additives. In addition to saline, potassium sulfate and sodium sulfate, mixtures of saline and sulfates can be used. Other suitable aqueous solutions include readily reduced oxidants such as solutions, gels, and solid-state materials containing at least a portion of ferric ion, triiodide ion, or ferricyanide ion, nitro-organics, aldehydes, soluble olefins, hydrogen ions (acids, including strong or weak acids), stannic oxide, lead dioxide, silver (I) and/or silver (II) oxide films, complexes, and salts; any noble metal oxide, including blends and including platinum group metals, nickel, and copper; halogens, including bleach (OCI⁻), OBr⁻ iodine, bromine (e.g., bromine water); bromate, iodate, periodate; quinine and other such reversible and easily oxidized or reduced quinine solutions and solid materials; cesium (IV) solutions and oxides; cobalt (III) solutions, complexes, and solid oxides; chromate (VI) solutions and materials; ferric tris (orthophenanthroline) complex in solution or solid form. For aqueous solutions and gels, electrolyte components can optionally include pH buffers. pH-buffering electrolytes that are effective are phosphate salts {MₓH_{y}(PO₄)_{z} for M=Na, K, and/or Li ions}, Goodes buffers, amino acid, carboxylic acid buffers and the like. Non-pH-buffering electrolytes can include brines and/or saline solutions consisting of alkali, alkali earth, ammonium, zinc ion, and the like including blends in any soluble combination, salts of sulfate ion, chloride ion, bromide ion, alkyl sulfonate ion, aryl sulfonate ion, nitrate ion, phenolate ion, and the like.

Outflow-1 253 is typically an aqueous solution and 254 can contain hydrogen generated in the cell, depending on the catholyte electrolyte selected. The hydrogen can be vented or otherwise disposed of. The divider 227 may be present or absent. When present, divider 227 is typically a membrane such as an ion exchange membrane or, in some embodiments, it may be a slotted or open pore divider that prevents mixing between the two volumes 231, 241 by flow control. Where the membrane is replaced by a simple divider, the divider may be simply a screen, preferably a fine screen that aids flow control.

In volume one 231, light 201 incident upon the catalytic active layer 215 that has passed from the waveguide 207 and through the first conductive layer 211 produces hydrogen peroxide by oxidizing water entering stream 251, the peroxide being received by stream 231 and is withdrawn as stream 253. The longer stream 231 remains in the cell, the more hydrogen peroxide accumulates in it, giving a higher H₂O₂ concentration in exiting stream 253. In this manner, flow rate of control of electrolyte 251 also controls H₂O₂ concentration in stream 253. Most preferred is to circulate stream 253 back to stream 251 to further build H₂O₂ concentration and to ensure good temperature control of the anolyte. Aqueous solution in outflow-2 254 is typically recycled to inflow-2 252 for efficiency and ease of use, but is optional. As noted earlier, a bias voltage is typically applied or developed across the electrodes that are labeled as V⁺ and V⁻. In another embodiment, a semi-conductive PN junction or diode that has an active layer that faces 215 can be used to polarize the first conductor and the active layer by the bias voltage so developed.

Referring now to FIG. 2B, a further embodiment of the invention provides for apparatus 200B wherein light 201 is incident on an antireflective coating 203A that is placed upon an end of waveguide 207. In some embodiments, as illustrated here, a second antireflective coating 208 may be placed adjacent to waveguide 207 and between waveguide 207 and first conductor layer 211.

Referring now to FIG. 2C, an additional embodiment of the invention provides for a protective layer 220 adjacent to the outer surface 216 of the catalytic active layer 215 and positioned between the catalytic active layer 215 and the aqueous electrolyte 251 in volume 231. The protective layer 220 serves to protect the active layer 215 from attack by the aqueous electrolyte 251. For metal oxide catalysts that quickly dissolve in acids, for example ZnO, typically the attack occurs as follows:

ZnO(solid film)+H₂O→Zn(OH)₂(surface)

Zn(OH)₂(surface)+2H⁺(from anolyte)→Zn²⁺(aqueous)+2H₂O

where Zn²⁺ (aqueous) indicates dissolved zinc ion and hence erosion of the photocatalyst.

Suitable candidates for protective layer 220 can be porous metal films, phosphatized layers of zinc or aluminum phosphates, or blends thereof, dopant metal ions such as TI (IV), Sn (IV), W (VI), Ce (IV), Al (III), Ca (II), Ba (II), rare earth ions Y (III), Ge (IV), B (III), and the like. Hydrated polymer films and gels can also be used to retard the solubility of ZnO and other readily dissolved films. The most preferred manner to prevent ZnO film dissolution in anolyte is through the use of an electrolyte containing pH buffers that control the pH in the range of 6 to 10.

Referring now to FIG. 2D, this figure illustrates a portion of a cell of FIGS. 2A to 2C where active layer 215 is replaced by two active layers, a first active layer 215A and a second active layer 215B. First active layer 215A captures photons provides charge separation while the second active layer provides for hydrogen peroxide production. As further discussed herein, this embodiment is useful where materials in the first active layer 215A are more efficient for photon capture and separation of electrons. This embodiment is also useful where the second active layer 215B is more efficient for hydrogen peroxide production than the first layer 215A. For example some materials such as TiO₂ tend to produce oxygen rather than activated oxygen or hydrogen peroxide. Examples of materials useful for the first and second active layers include those metal oxides and the like given herein. An optional porous protective layer 217 may be used as under conditions that are corrosive for the second active layer 215B as further discussed herein.

Referring to FIG. 2E, the photolytic hydrogen peroxide generator 110 pumps aqueous solution or electrolyte from a treatment volume to the photolytic hydrogen peroxide generator 10 through an inlet 12. The aqueous solution enters by means of a flow distributor 25 into one or more photolytic cell(s) 16. The photolytic cell(s) may be optionally arranged to form a stack of photolysis cells 27. The amount of aqueous solution entering and leaving the photolytic cell(s) 16 is controlled by flow rate to distributor 25.

A light source 20 irradiates the photolytic cell(s) 16, thereby initiating the photochemical reactions within the photolytic cell(s) 16 that ultimately form activated oxygen. Optional hydrogen gas formed from the cathodic chemical reactions in the photolytic cell(s) 16 enters one or more gas separation devices 24 for eventual venting trough a venting outlet 28, or is allowed to diffuse through gas diffusing liquid carrying tubing. Once the aqueous solution contains activated oxygen, the aqueous solution returns to the treatment volume via outlet 22 and/or is recirculated to stream 12 for further concentration. Among the components of the photolytic hydrogen peroxide generator not illustrated in this embodiment is the pump, power supply, control electronics and sensory technology for monitoring reaction chemistry, the amount of activated oxygen, hydrogen gas, etc., generated the presence of chemicals and/or potential toxins, etc. Note that a pump is required for the case of electrolyte recirculation.

Referring now to FIG. 2E, the individual cells of the invention can be stacked up and linked together, normally in parallel form to form a cell stack. The design allows illumination of each photolytic cell in the stack. Only one or a few electrolyte entry and exit points are needed. Any gases exit with the flowing electrolyte and are removed using a gas/liquid separator device of well known design.

The main component of the photolytic hydrogen peroxide generator is the photolytic cell 16. See, for example, FIGS. 2A to 2F. Referring now to FIG. 2F, light energy 21 from a light source 20 enters the photolytic cell 16 through a transparent window 30, penetrates conductor 26, and activates a layer of light-activated catalyst 32. Any light reflected back into window 30 is due to the ATR film on 30. As discussed in more detail below, an example of such a light-activated catalyst is a metal oxide(s) such as ZnO. Depending on the catalyst 32 used, the light-activated catalyst 32 converts water into activated oxygen, hydrogen ions, and electrons. Materials 34 that promote reactions that produce oxygen as O₂ (dissolved or gas) from the activated oxygen are to be avoided in production of H₂O₂. An example of a material 34 to be avoided is manganese dioxide (MnO₂) and other metal ions readily capable of redox cycles such as iron (III/II), cobalt (III/II), silver (II/I), or copper (II/I). Note that films 34 and 220 are the same.

Electrons are formed during the conversion of water to dissolved oxygen and are conducted out from the catalyst 32 to an anode conductor layer 26 such as indium-tin oxide (ITO), gold or titanium thin metal film or screens. In chamber 37, the hydrogen peroxide enters the aqueous solution and flows to the treatment volume via outlet 22.

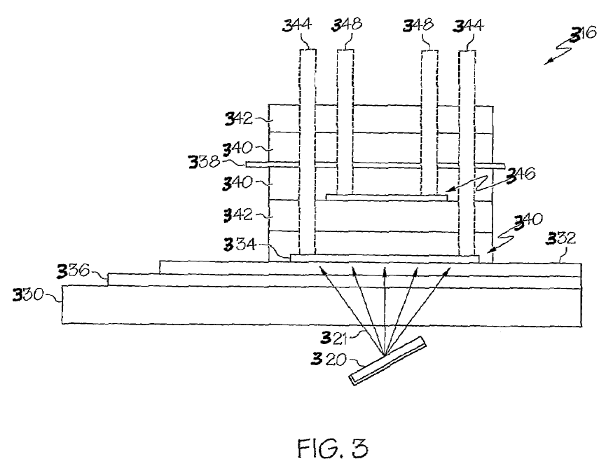

FIG. 3 shows a flow-through a flow-through embodiment of the photolytic cell 316. In the flow-through cell embodiment, the following main components of the photolytic cell 316 are assembled, i.e.; a conductive coating of vacuum-deposited electrical conductor 336, a coating of adherent ZnO 332, an optional sealing layer 334. Layer 334 is used to reduce or substantially prevent ZnO from being dissolved in the aqueous solution. A UV laser light 320 impinged on the transparent glass or quartz substrate so as to initiate the reactions. As discussed below, this cell was utilized to collect pH and electrical current data as a function of laser UV irradiation demonstrating critical components of the invention.

In this regard, photolytic cell 316 of FIG. 3 includes a transparent window 330 or wave guide for the entry of light energy in the form of photons 321 from a light source 320 such as an ultraviolet laser light. On one side of the glass slide is an anode conductor layer 336, such as Au, ITO, Ti, Cr, or other metal film. Attached to the anode conductor layer 336 is a layer of a light-activated catalyst 332 such as TiO₂, platinized TiO₂, and preferably ZnO. An optional material to control metal oxide dissolution in the aqueous solution, such as sealing layer 334, such as porous metal films, phosphatized layers of zinc or aluminum phosphates, or blends thereof, dopant metal ions such as Ti(IV), Sn(IV), W(VI), Ce(IV), Al(III), Ca(II), Ba(II), rare earth ions such as Y(III), Ge(IV), and B(III), and the like. Porous films, plates, and gels also can be used to retard the solubility of ZnO and other readily dissolved films. The most preferred manner to prevent ZnO film dissolution in anolyte is through the use of an electrolyte containing pH buffers that control the pH in the range of about 6 to about 10 is adjacent to the light-activated catalyst layer 332. The photolytic cell 316 of FIG. 3 typically includes one or more layers of silicone gaskets or spacers 340 and an acrylic housing 342. A pair of anolytes 344 (in/out) is connected to the light-activated catalyst layer 332 or optional catalyst layer 334 and extends through the photolytic cell 316 away from the transparent window 330. The photolytic cell 316 further includes an optional cation exchange member 346 such as NAFION™ membrane. A pair of catholytes 348 (in/out) is connected to the cation exchange member 346 and extend outwardly through the photolytic cell 316 generally away from the transparent window 330. The photolytic cell 316 further includes a cathode electrode 338, such as Pt, stainless steel, or nickel foil, adjacent to the silicone spacer 340. The operation and use of this embodiment of the invention is more particularly described in the Examples below.

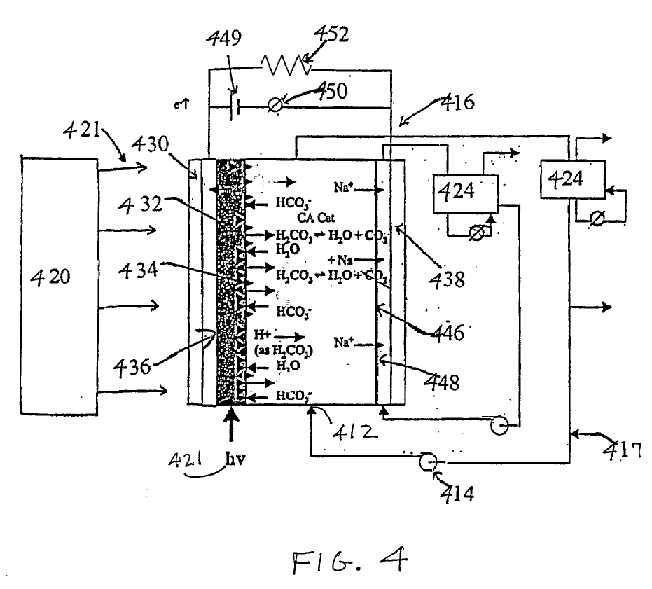

FIG. 4 is a schematic drawing showing the electrical and chemical transformations which occur in the photolytic cell 416 of the photolytic hydrogen peroxide generator. Aqueous solution low in activated oxygen and from a treatment volume, from fresh solution enters the photolytic cell at inlet 412 and/or being recirculated from stream 417, through inlet 412 by way of a pump 414. Light photons (hv) 421 generated by light source 420 enter through a transparent window 430 or wave guide 418 and activate the light-activated catalyst 432 such as 0.4-200 μm ZnO film. Depending on choice of metal oxide catalyst used, the light-activated catalyst 432 either converts water to activated oxygen and/or converts water directly to hydrogen peroxide at the anode-electrolyte interface 434.

The electrons released from the conversion of water to activated oxygen are collected in the transparent collector electron anode 436. An electrical positive voltage applied from a supply of electrical DC energy such as battery 449 or DC power supply directs the electrons to flow from the anode 436 to the cathode 438, such as graphite or nickel, so that the electrons do not react with the activated oxygen to cause a back reaction.

The photolytically generated electrical current and electron flow can be monitored by a current meter 450; an optional external load 452 (this resistance can be a load to do useful processing, for example, operate a pump for a liquid and/or vapor/gas pump, or to energize electronic controls, and the like). The catholyte 448 can be the same or different than the anolyte, or could be spent anolyte requiring regeneration at the cathode 438. Depending on catholyte and anolyte compositions chose a ion exchange, anion or cation, membrane or a micro-porous separator 446 can be inserted to keep the two half cells separate, making the system a divided cell. Tanks and pumps 424 represent receiving surge or product tanks for the catholyte and anolyte. Electrolyte levels are monitored by electronic or visual inspection of the liquid levels in these tanks.

It is one aspect of the invention that the electrical current caused by these electrons can be adjusted to a voltage sufficient to drive such mechanical devices and/or to drive desirable electrochemical reactions at a cathode electrically connected to the photoanode described elsewhere in this application. The voltage that can be developed at the cathode is determined by the sum of the voltage of the exciton minus any IR drops across the electrical circuit to the cathode and through the cell to the photoanode to complete the full circuit. The voltage at the cathode is also determined by, and can be controlled by, the energy of the photons illuminating the photocatalyst where the greater the energy per photon the greater the voltage developed within the circuit, although the response is not necessarily linear. In addition the voltage can be boosted by hooking the photolytic cells in series, while the current flow can be boosted by linking the photocells in parallel. Both current and voltage amplification can be accomplished by configuring a group of photolytic cells in a pattern consisting of both parallel and serial arrangements. A DC amplifier can also be used to amplify the voltage. Additional amplification can also be achieved through further illumination too. The amount of voltage amplification needed is determined by the voltage required for the electrochemical reaction desired at the cathode and the internal IR drops around the circuit. Electrochemical reduction (cathodic) reactions involving low voltages and that do not have significant overpotentials will require very little or no additional amplification and this is reflected in the high current found in the cell circuit, provided internal IR drops are minimized through the use of good electrical contacts, highly conductive electrolyte, and low IR drops across the internal membrane if a membrane is present at all. As the voltage requirements increase by selecting more and more demanding electrochemical reduction chemistries, then multi-stacking the photocells become more desirable to achieve fast cathode production rates. We note too, that choice of cathode electrode materials need to be chosen matched to the chemical reduction desired to promote small overpotentials and IR drops.

Suitably designed with sufficient voltage and described above, the electrons derived from the photocatalyst can be directed to react with water, acids, or pH buffers to form hydrogen gas, H₂, or other electrochemically reducible chemical species, at the cathode. Preferred reducible species are those listed previously (see for Inflow-2 252, above). When hydrogen gas is formed, it is moved to a gas separation or sorption device, where it is collected and/or released or collected for use as a co-product. Cations, preferably sodium (Na⁺) ions or H⁺ ions, from the aqueous solution (anolyte) migrate across the cation exchange membrane 46 and react with hydroxyl ions to form sodium hydroxide (NaOH) in the catholyte 48. The hydrogen ions formed from the conversion of water at the light-activated catalyst also diffuse via the well-known rapid “proton hopping” mechanism through the separator (either a cation exchange membrane, a proton exchange membrane (PEM), a fine screen, porous frit, or porous ceramic, alone or in any combination) to the cathode, where it is converted to H₂ or where it participates in another cathodic reaction, or where it remains as hydronium ion {H⁺(aq), H₃O⁺(aq), or simply H⁺} as part of the acid forming there by electrochemical reduction of other chemical species forming anions. As an example, bromine water, containing Br₂ in water, can react at the cathode to form bromide ions, Br(aq), that, when associated with the H⁺ ions from the anolyte section, form hydrbromic acid, HBr, solution, vapor, or gas. Other examples are oxone-forming sulfuric acid, triodide ion-forming HI, or other (see the list for Inflow-2 252 above) candidates for electrochemical reduction and simultaneous or follow-on association or protonation with H⁺ ions). If gas is produced at the cathode generator it is moved to one or more gas separation devices 24, vented, or used in a subsequent reaction or application. The aqueous solution containing activated oxygen exits the photolytic cell 16 via an outlet 17 and returns to the treatment volume.

The various particular components and/or processes of the present invention are described in more detail below.

### 1. Transparent Window 30

The transparent window 30 can be formed from plates, thin films, or coatings of glass, quartz slides, quartz, fused silica, silica gel, clear plastic, etc. Glass is useful in forming the transparent window provided that the UV transparency is adequate at the wavelength needed. Quartz slides, films, or plates are also useful because of their high UV transparency. For the transparent window, light entry into and through the window can be from the back, side, edge, or bottom. Edge illumination through the transparent window can include a lens or wave guide(s). For low or non absorbing electrolytes the illumination can be accomplished through the electrolyte. In this manner simpler device designs are possible as is heat control. Through the electrolyte illumination is the most preferred configuration of the invention since an opaque current collector can be used and the cathode is more easily arranged since it can back up to the photo-anode.

The transparent window can further include a wave guide. A wave guide uniformly supplies and/or distributes photons (hv) from the light source over at least a portion of the surface of the light-activated catalyst. Most preferably, the wave guide causes the light photons to travel in a path so that the photons maximally contact the entire layer of the light-activated catalyst. Light enters the wave guide in the side of the transparent window generally parallel to the surface of the light-activated catalyst that is attached to the transparent window. The wave guide allows for maximal light photon contact with the light-activated catalyst without directly illuminating the side of the entire light-activated catalyst attached to the transparent window. The wave guide also allows for maximal photolytic cell stacking because light is not required to directly illuminate the light-activated catalyst, but, rather, can be indirectly illuminated by side or edge entry in the transparent window. The wave guide provides additional efficiency to light used in the photolytic cell because the light can be spread across the entire surface of the light-activated catalyst.

### Anode Conductor Current Collector Layer 36

The anode electrical conductor layer 36 conducts electrons released into the photo catalyst conductance band from the charge separation formed on photon absorption. The anode current collector conductor layer and associated internal bias voltage, external bias voltage, and/or the cathodic reaction polarization of the cathode prevent the electrons from back-reacting with the activated oxygen, including H₂O₂ produced at the water interface with the photocatalyst 18, to reform water, thereby allowing maximal formation (maximal quantum yield) of activated oxygen and H₂O₂. The anode conductor layer is preferably a thin film or coating applied, formed, or otherwise intimately connected to the photocatalyst. If the illumination is to be performed through the current collector layer then it needs to be at least partially transparent over at least a portion of the electromagnetic spectrum corresponding to 190 nm to 750 nm. In this case the current collector is physically intimately attached to at least one side of the transparent window, and preferably to both sides. Most preferably, the transparent window also contains an anti-reflective coating to further increase quantum yield efficiency.

The anode conductor layer can be formed in a number of conventional manners. A description of two different ways follows. The anode layer can be formed by attaching a thin film or grid or lines of uniform metallic or semi-conductor material to the transparent window using one or more well known techniques such as vapor deposition, electroless metal plating, or vacuum sputter coating, and the like and with or without photoresist pattering to make grids. Such grids must be electrically continuous to enable any electrons collected therein to flow to the external or internal circuit and thus allowing the film to perform as a current collector. The film preferably has a thickness of less than about 0.2 μm. Preferably, the film is formed from a noble metal, graphitic carbon, copper, tin, silver, gold, platinum group metal, indium tin oxide semiconductor, titanium, stannic oxide, gallium nitride, a metal, and the like alone or in any combination. Most preferred are metals that, when oxidized, is photolytically active in forming activated oxygen and/or H₂O₂, for example titanium, zinc, gallium, cadmium, and the like. Gold remains metallic at all conditions, but can be very efficient at UV light blockage or reflection, and so would be most effectively used as a screen or grid to allow light passage through the openings. Typical metals and semiconductors useful in this regard include Pt, Ni, Cu, Ag, Au, and In—Sn oxide (ITO), and the like, and the metal oxide film formers: Ti, Zn, Cr, W, Al, and the like.

An example of using a metal to form both the electrical conducting layer 36 and the photocatalyst 18 follows. Zinc can be oxidized to ZnO by exposure to air or, most preferred, by adding O₂ to the vacuum deposition chamber during zinc metal sputtering or during chemical vapor deposition to yield a catalyst layer with excellent adhesion. Ti/TiO₂ and W/WO3 dual films, and well as blends of these metals, alone or in combination with dopants and/or dye sensitizers can also be prepared in the same manner.

The anode current collecting conductor layer 36 can also be formed by using photo-resist technology. Under photo-resist technology, grids are prepared with photosensitive photoresist organic materials and masks using vapor deposition. These resists are applied then exposed to UV to cure a pattern into the resist, then are developed into a pattern of mask alone, for example, lines, grids, or screens, by exposing the mask to UV curing radiation, the removing any uncured photoresist material by dissolution with a reagent, and then metal plating or sputter coating onto the cured pattern. Such thin film processing methods are well-known in the prior art and referred to as integrated circuit fabrication (IC FAB) operations. Conductor line spacing, width, and thickness optimization and matching to the light wavelength range is most preferred to minimize light attenuation, and to provide sufficiently close electric field effect on the photocatalyst film, good electrical connection to the photocatalyst semiconductor material, to provide electrically conductive areas to sweep electrons away from the adjacent light-activated catalyst layer 18.

### 3. Catalysts 32 and 34

One or more light-activated catalyst 32 layers are coated onto the anode conductor layer. In use, the light-activated catalyst is photochemically illuminated from any direction, as described above and below, whereupon it reacts with water to form activated oxygen intermediate that is ultimately converted to hydrogen peroxide H₂O₂. The term “activated oxygen” in the present application defines any free atomic, peroxide, oxygen with a valence of one, ozone, hydroxyl free radical, superoxide, singlet oxygen, or radical oxygen intermediate formed in the photolytically energized reaction of the photocatalyst in contact with water that is most preferably ultimately converted to peroxide, peroxide anion, HO₂⁻, or hydrogen peroxide. The activated oxygen formed is in the form of a peroxide, including one or more of H₂O₂, peroxide ion salt, hydroxyl free radical, superoxide ion, singlet oxygen, etc. or blends and mixtures of these, and is converted into hydrogen peroxide spontaneously at the water or water vapor interface. However any and all of these active forms of oxygen are effective for sterilization or etching applications. The amount and type of active oxygen and of peroxide formed depends on the light-activated catalyst used and on the electrolyte (anolyte) composition used. Also, depending on the light-activated catalyst and electrolyte used, water may be most preferably photolytically converted directly into hydrogen peroxide without first forming significant amounts of other activated oxygen intermediates.

Several different catalysts can be employed for producing hydrogen peroxide photochemically. One catalyst that can be used to photochemically produce hydrogen peroxide is zinc oxide without or with additives, dopants, dye sensitizers, and/or a sealing coating. By using zinc oxide, H₂O₂ is produced directly from water at a pH of about 6 to about 8. H₂O₂ is an excellent form of activated oxygen for providing sterilization, etching, oxidation, or other uses such as for Fenton's reagent or for DNA fingerprint analysis (genetic testing). Zinc oxide film has other positive attributes including known film formation technology, can be prepared in either vacuum or open air production environments (e.g. via the zinc/nitrate/glycine reaction and the like, or vacuum sputter techniques), high H₂O₂ yields, low toxicity concerns, and low cost.

Another example photocatalyst material that can be used to photochemically produce hydrogen peroxide is tungsten oxide (WO₃) that is exposed to visible light and using e⁻_{scb} removal. WO₃ tends to yield oxygen (O₂) directly from water without the need to first produce an activated oxygen species and so only yields H₂O₂ in low yield unless it is first coated with a second film of H2O2 forming oxide, for example ZnO or one of the oxides of Table 1, or a blend of these. As before, WO3 can be suitably alloyed with other elements, dopants, dye sensitizers and achieve enhanced yields and quantum efficiencies by using conditions such as acidic anolyte/electrolyte and/or readily reduced solute-containing electrolytes. WO3 is preferred as these multi-layer constructs since only visible light is needed to generate H₂O₂ from WO₃, especially if doped, at wavelengths than about 496 nm. In another benefit WO₃ films present low toxicity concerns. Preferably, the use of WO₃ or any other photocatalyst further includes the removal of excess e⁻_{scb} formed during H₂O₂ formation from water using slowly reduced redox reagents such as acidic ferric ion, ferrocene derivatives, ferrocyanide/ferricyanide, triiodide/iodide, bromide/bromate ion, quinoline/8-hydroxy quinoline, ferroin, tris(orthrophenanthrolene) iron(II)/iron(III), ruthenium complexes of pyridine-based complexes, and the like, as described previously.

Other catalysts suitable for reacting with water to produce H₂O₂ are given in Table 1 under UV/VIS radiation, in which a current collector anode removes the e⁻_{scb} efficiently from the production area in order to ultimately obtain good H₂O₂ production rates and fluxes and to minimize any back-reaction to reform reactants. The removal of e⁻_{scb} is performed through electronic conduction via the semiconductor property of the ITO current collector with enhancement via application of a small DC bias voltage using one or more of the following; a PN junction (for example located at the interface between the photocatalyst and the current collector, a diode, an applied external bias DC voltage, a facile cathodic reaction, and the like.

Irradiation of the materials in Table 1, alone or in any combination, produces H₂O₂ and most if not all also presents low toxicity concerns.

Most preferably, pH control and maintenance using for example pH buffers or concentrated acid or basic compounds, enhances insolubility and kinetic inertness to minimize dissolution and fouling during use and maintenance. Such pH regions of stability exhibited by metal oxides and metal hydroxides, Preferably, UV light is chopped or pulsed during photocatalyst irradiation to allow time for the chemical reactions to occur, since continuous irradiation may cause the e⁻_{scb} to accumulate and force a back-reaction with H₂O₂ to form water. A pause in the irradiation allows time for the slower, but still extremely fast, irradiation in the range of about 1 sec to 1 msec to occur.

A further catalyst for reacting with water to ultimately form H₂O₂ is a semiconductor powder (SCP)-filled UV/VIS light transparent thermoplastic film. SCP-filled thermoplastic film is relatively inexpensive to manufacture and to form into shape. SCP film is easily moldable, extrudable, cut, and machined. SCP can be used very efficiently in surface-applied-only form. Also, SCP has low toxicity concerns and is stable over a broad range of pH. Optimized commercial products (conductive plastic filler powders) are also available and these possess good properties for dispersion, particle-to-particle electrical conductivity (for e⁻_{scb} removal), low, neutral, and high pH resistance, and resistance to sloughing off that can be used with the present photolytic hydrogen peroxide generator.

The following additional preferred conditions may be used for each of the above-mentioned catalysts. First, an application external or internal to the cell of a small (e.g. about one volt, but can be up to a few volts DC and as low as a tenth of a volt, or even as low as hundredths of a volt). This bias voltage can be optionally applied to help ensure that the e⁻_{scb} is quickly conducted away from the production site. This bias voltage works by charging the anode, which then forms an electric field across the photocatalyst, thereby directing the negatively charged electrons to the current collector. Preferably, less than 1 volt is used; most preferably, far less than 1 volt where 0.1 volt is most preferred, and 0.01 volt is most preferred. Also, when the cathodic reaction is rapidly reversible at the voltage supplied by the photocatalyst anode, addition of a bias voltage application may be superfluous. The more conductive the photocatalyst, and/or the more facile the reduction chemistry at the cathode, the lower the bias voltage that is effective for electron collection.

Second, a chopped illumination, instead of a continuously applied illumination, may be optionally used to avoid the occurrence of unwanted secondary chemical reactions by electron concentration accumulation in the photocatalyst, especially electron-hole recombination, reduction of active oxygen, or reduction of H2O2 product back to water. It is believed that this enhancement is possible since the secondary chemical reactions are far slower than the photochemical reactions and the removal of exciton components enhances photolytic yields by allowing the excited electrons to exit the system and so not be present for regeneration of starting material from activated oxygen or H₂O₂, to reform water. In addition, at very high photon flux intensities, and interlude insures that sufficient electronic ground state catalyst material exists for high photon absorption factors than in turn increase quantum yield that in turn reduce lamp size and associated power supply.

Photocatalyst systems such as zinc oxide (ZnO) or the other materials of Table 1 and the like are selected such to preferentially release hydrogen peroxide as the activated oxygen more readily than do other photocatalysts, such as TiO₂ or WO₃. or the other catalysts of Table 2 and the like. Although we do not wish to be bound by any theory, this selective H₂O₂ production capability is understood as follows. Less acidic metal ions under the Lewis acid/base theory definition, such as the materials of Table 1, cannot sufficiently stabilize the highly alkaline peroxide anions, either O₂²⁻ or HO₂⁻, relative to protonation by water (pKₐ₁ of H₂O₂ is 11.38 at 25° C. while pKₐ₁ of H₂O is 14.0 at 25° C.) at the surface of the solid photocatalyst phase, and so hydrogen peroxide, H₂O₂, is readily formed from the materials of Table 1, for example, for ZnO.

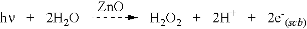

ZnO films and particles can be prepared in a number of ways with varying but controlled composition, morphology, thickness, and porosity. For example, mirrors of zinc, doped zinc, and zinc alloys can be sputtered down onto an optically transparent support, followed by oxidation with O_{2(g)}. This treatment produces a metal/metal oxide (Zn/ZnO) dual film. Another highly effective approach to prepare semiconducting ZnO-based films is to utilize a process for forming ZnO films on surfaces including optical glass in the open air. (L. R. Pederson, L. A. Chick, and G. J. Exarhos, U.S. Pat. No. 4,880,772 (1989)) The optical glass coating technique is based on applying a zinc nitrate/glycine aqueous solution as a dip or spray, followed by drying (110° C. for 15 minutes), then heating (450 to 500° C. for 3 minutes) to initiate a exothermic self-driven oxidation reaction during which the carbon and nitrogen exit as gases, leaving an adherent yet porous ZnO film bonded to the underlying surface (e.g. glass in this example) and is referred to as the glycine nitrate process (L. R. Pederson, L. A. Chick, and G. 3. Exarhos, U.S. Pat. No. 4,880,772 (1989)). The ZnO film is normally produced doped with alumina by including aluminum nitrate in the aqueous formulation for the initial dip. Many other metal ion blends are also possible with this technique as described in the referenced patent and these are included in this application by reference.

The advantage of tungsten oxide, WO₃, is that it only requires visible light to produce H₂O₂. However, WO₃ tends to produce oxygen directly without requiring a second catalyst to form dissolved oxygen. The lower photon energy requirement for WO₃ is due to the smaller band gap of 2.5 eV versus at least 3.2 eV for TiO₂₍ₐ₎. As with the TiO₂ anatase system, high yields are possible with the WO₃ catalyst if the e⁻_{scb} electrons are removed. To produce H₂O₂ with these refractory metal oxides photocatalysts, (Table 2 and the like, for example, WO₂ TiO₂, and the like), and not use a second layer from one or more of the oxides of Table 1, requires the anolyte to be acidic, preferably pH<4, and more preferably pH<2, and most preferably pH<1. In this manner the peroxy species is protonated as it forms on the surface of the catalyst to form and release the H₂O₂ prior to its disproportionation to O₂.

These refractory metal oxide photocatalysts (Table 2 and the like) can be coated with a H₂O₂ producing metal oxide catalyst second layer (selected from Table 1 and the like) that then can accept h⁺ moieties from the first photocatalyst film layer, which then enables H₂O₂ production at neutral to slightly alkaline pH from the second layer catalyst (selected from Table 1 or the like) as the h⁺ moiety reaches the catalyst layer 2/water interface or catalyst layer 2/porous sealer/water interface. For example pH 6-9 electrolyte or water is employed for ZnO 34 second coated layer placed on the surface of TiO₂ film 32 first layer that is applied to the electronic conductor film 36 (FIG. 4).

An advantage exists when the H₂O₂ producing film is a filled plastic. Such materials are often rugged, inexpensive, and manufactured easily. Commercial sources exist for semi-conducting, low light absorbing, inorganic fillers for plastics which are supplied in ready made condition for incorporation into plastics, making the plastics electrically conductive. For example, E.I. duPont Nemours, Inc. sells electroconductive powders (EPC) under the trade name ZELEC® ECP for such purposes. The conductive substance in ZELEC® ECP is antimony-doped tin oxide (SnO₂:Sb). The bulk of these materials, onto which the conductor is coated, are familiar inorganics such as mica flakes, TiO₂, and hollow silica shells, or ECP-M, ECP-T and ECP—S respectively. Pure SnO₂:Sb-based material is designated ECP—XC and is a much smaller particle than the other materials. About 25-45% by weight of the ECP products are used so that the particles are sufficiently close to each other to provide internal electrical connections throughout the otherwise non-conducting plastic. ECP-S and ECP-M normally perform best for lower concentrations. Thin films of ECP-XC can provide an attractive coating because they are very fine grained and strongly light absorbing. As plastic films are often transparent to visible light and UVA, but not so transparent to UVB or UVC light, it is most preferred to apply plastic-based photocatalyst constructs when including dye-sensitized photocatalyst systems as these dyes enable the use of the entire visible spectrum. In these cases the dye absorbs a visible or UVA photon and then ejects an electron into the normal photocatalyst conduction band which then loses it to the current collector. The dye then replenishes its electron from the electrolyte (water or redox active solute).

The TiO₂ layer mentioned above can be formed a variety of ways. The TiO₂ layer can be formed by sol gel, drying (then room temperature or thermal curing or sintering). A product under the trademark LIQUICOAT® from Merck & Co., Inc., which hydrolyzes titanium alkoxide, Ti(OR)₄, type material in water to form TiO₂ and 4ROH can be used to form the TiO₂ layer under a sol gel/drying/curing process. TiO₂ can also be formed from preparing an anatase suspension from dry powder, then dipping, drying, and curing the suspension to form the TiO₂ layer. Another way the TiO₂ layer can be formed is by e-beam evaporating titanium metal and subsequently exposing the titanium to O₂ within a deposition chamber. The TiO₂ layer can also be formed by adding titanium salt to water and adjusting the pH to ˜2-7 to form a suspension, then dipping (construct (FIG. 2D, layers)) the suspension and allowing the suspension to dry in the air or oven to a film.

Activated oxygen is created from TiO₂ by irradiation with UV light, but the chemical form of the activated oxygen is very reactive and can be lost by side reaction occurring in close proximity to the TiO₂ particle surface where activated oxygen is generated. To minimize the loss of activated oxygen to unwanted side reaction, and instead promote the formation of H₂O₂, move the activated oxygen to H₂O₂ conversion point closer to the activated oxygen generation point, i.e. move the metal ion catalyst film for H₂O₂ formation (Table 1) as close as possible to, that is, in contact with, the TiO₂ film.

The amount of activated oxygen lost by side reactions can be minimized by introducing an activated oxygen carrier molecule into the media, or “D,” by analogy to a photosynthetic system. Agents for use with species D can be selected from those that readily form organic peroxides such as carboxylic acids or alcohols. Organic peroxides are useful because they readily can be converted to H₂O₂ and readily form by oxygen insertion. The organic peroxide reactions are as follows:

2(TiO₂)—Ti—O₂+nhν→2e⁻+((TiO₂)—Ti—O—O—Ti(TiO₂)²⁺ (2)

where {TiO₂} indicates the bulk TiO₂ film, and —TiO₂ the point of hν absorption, where the excited electronic transition corresponds to a ligand-to-metal charge transfer (charge separated electron-hole (e⁻-h⁺) pair or exciton), and is followed by the following reactions. By electron exchange the Ti^{IV}-peroxide “hole” migrates to the metal oxide catalyst surface (surface of the only layer or the second layer as appropriate) and adjacent the water of H₂O vapor condensate where H₂O₂ can form by proton transfer, electron transfer and/or O-atom insertion reaction. The peroxo species represents an example of the “hole” or “activated oxygen” referred to earlier. This species can lead to H₂O₂ either one or two ways; either directly,

((TiO₂)—Ti—O—O—Ti(TiO₂))²⁺+H₂O→2(TiO₂)—TiO₂+H₂O₂+2H⁺ (3)

or indirectly (using carboxylic acid example);

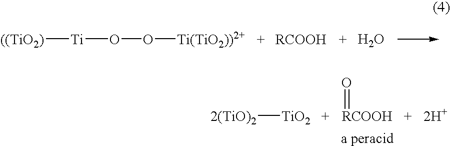

Where uninvolved other ligands of Ti are not shown. The peracid can then be used as is for disinfection or oxidation, concentrated and used, and/or converted to H₂O₂ by hydrolysis, for example.

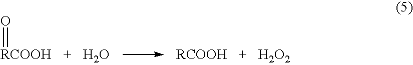

where conduction of the e⁻ into the semiconductor conduction band and away from the location of the “hole” component of the exciton prevents recombination of hole with the e⁻ which would result in not net change other than some heating and a lowering of quantum yield. As shown in the reaction above, the TiO₂ anatase is regenerated in Reaction 3 or 4. The above reaction produces a hydrogen ion, H⁺, that is useful for other uses, is neutralized by the buffer, which in turn can be regenerated at the cathode, or the H⁺ can be reduced to H₂ at the cathode to form a useful product or that can be released as waste.

The catalyst candidates that cause the conversion of the activated oxygen into O₂ gas or to dissolved oxygen, and hence are undesirable for the current invention, includes metal ions capable of redox cycling, such as Fe^{II}, Fe^{III}, Cu^{I}, Cu^{II}, Co^{II}, Co^{III}, Mn^{II}, Mn^{III}, Mn^{IV}, Ag^{I}, Ag^{II}, and the like, or metal oxides formed from metal ions capable of redox cycling, such as manganese dioxide, MnO₂, Fe₂O₃, and the like. The present reaction produces dissolved oxygen directly from water and by-passes the gaseous state. The MnO₂ catalyst is most preferred because it forms dissolved oxygen efficiently and is not highly selective of the activated oxygen form.

### Cation Exchange Membrane 346

The optional cation or anion ion exchange membrane 46 allows for the diffusion of anions or cations in the photolytic cell. Particularly, the cation exchange membrane allows a cation, such as a sodium ion (Na⁺), hydrogen ion (H⁺), hydronium ions (H₃O⁺_{(aq)}, potassium ion, ferric ion, ferrous ion, lithium ion, alkali metal ion, alkaline earth ion, silver ion, (Ag⁺), protonated ammonium ion (NH₄⁺) and ammonium derivatives (R₃NH⁺, where R═H, alkyl, alkylaryl, or aryl, including where R groups are the same or different), phosphonium ion, quaternary ammonium ion (R′₄N⁺, where R′ cannot be H, and where R′ is alkyl, alkylaryl, or aryl), where R and R′ can include nonhydrocarbon groups such as alkoxy, alcohol, hydroxyl, ether, keto, halide, cyano, nitro, mercapto, thiol, thioether, phosphonate, amino, and the like groups as inert components or means to improve membrane permeability, to control degree of hydration, to control solubility, to affect oxidation/reduction potential and the like. These cations are derived from the substantially or completely aqueous solution or gel electrolyte to diffuse through the membrane and subsequently form sodium hydroxide (NaOH) in the catholyte. The cation exchange membrane is commercially available under the trademark NAFION™ and is available from E.I. du Pont Nemours Inc. NAFION™ cation exchange membranes are a perfluorosulfonic acid/PTFE copolymer in an acidic form. Although NAFION™ cation exchange membranes are the preferred membrane, one skilled in the art would recognize that other cation exchange membranes are also suitable in the photolytic cell. H+ and K+ ions and the like also migrate.

### Anode

In certain cases the photolytically derived oxidized species or cation will be complexed or chelated with anions, for example ferric or ferrocyanide ions, in which even the membrane required may need to be an anion exchange membrane. Such membranes contain a high density of quaternary ammonium and/or phosphonium groups held within a porous, polar, hydrated membrane. In addition, neutral, porous membranes, gels, frits, ceramic devices can be sufficiently effective.

The anodic compartment of the photolytic cell has the series of reactions previously described.

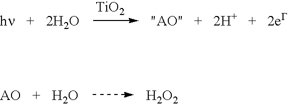

In one embodiment of the invention, the electrons formed in the anodic reaction are conducted away to a cathode via the anode conductor layer, grid, or wire. The cations charged and/or oxidized species, for example Na⁺ ions and/or hydrogen ions are moved to a catholyte via a cation exchange membrane, gel, or porous frit described above.

### Catholyte 48

When the electrolyte is a sodium salt pH buffer, and the cathode reaction is water reduction (H₂O+e⁻→½H₂+OH), sodium hydroxide (NaOH) builds in the catholyte during the series of reactions in the photoelectrochemical cell. It is preferred that the NaOH, a useful material, is purged occasionally from the catholyte. If sodium chloride (NaCl) is used in the anolyte, OH will eventually form in the catholyte and would periodically be purged.

An example of the reactions occurring in the cathode of the photolytic cell when Na₂CO₃ aqueous solution is the anolyte and catholyte, or an electrolyte in the case of an undivided cell are as follows:

Na₂CO₃ (received from cathode compartment)

Catholyte is NaHCO₃ (received from anode compartment)

Cathode immediate product is NaOH and H₂, i.e.

2H₂O+2Na⁺+2e⁻2NaOH+H₂(g)

Anode Immediate material product is H+, i.e. 2H₂O+hν→H₂O₂+2H⁺+2e⁻
(The immediately above reaction takes place in the presence of a photocatalyst)

Anolyte reaction is:

Na₂CO₃+2H⁺→2NaHCO₃

Catholyte reaction is:

NaHCO₃+NaOH→Na₂CO₃+H₂O

Over all reaction of cell:

2H₂O+hν→H₂O₂+H₂(g)

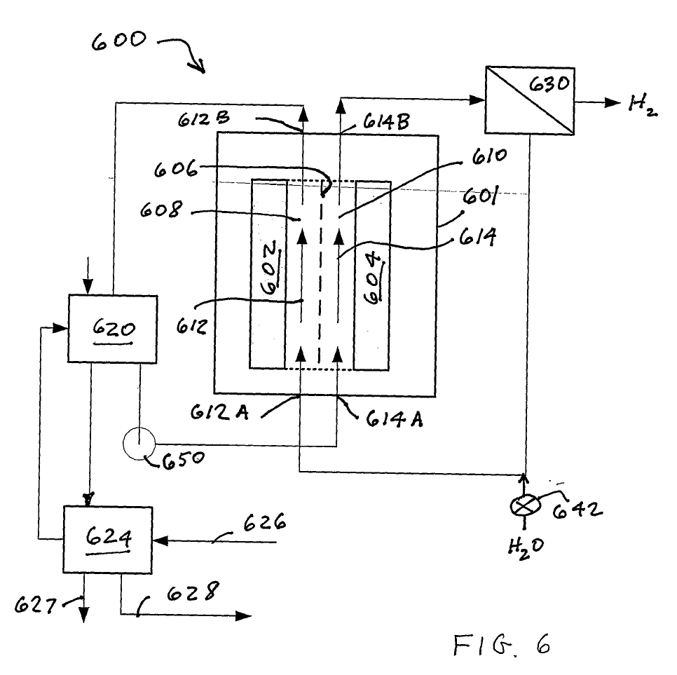

Hence the Na₂CO₃ consumed at the anode is regenerated at the cathode. Hence, during the course of operation the catholyte is pumped through the catholyte compartment and upon exiting the cathode compartment for regeneration (FIG. 6. In this manner electrolyte replacement is only seldom needed to remove any accumulated impurities. Water makeup, preferably using purified water, is added as needed to replenish the water consumed making H₂O₂ and H₂ (see above equation). Notice that the above reactions require a cation (Na⁺ in this example) to traverse from the region of the anode to the region of the cathode. If a divided cell or frit-partitioned cell is used, then this cation (Na⁺ ion exchange or diffuse respectively during the process. FIG. 6 is an illustration of operation of an H₂O₂ generation cell. Note that only a single cell is shown but that multiple cells (a “cell” stack) is also possible and preferred.

Referring again to FIG. 6 another apparatus for producing hydrogen peroxide 600 includes a cell wall. The cell contains a photo-anode 602 where the hydrogen peroxide is produced and a cathode 604. If desired, an optional flow separator 606 (my be a membrane in some embodiments) may be used. The membrane divides the cell into a anolyte compartment 08 and a catholyte compartment 610. Anolyte flow 612 and catholyte flow 614 are preferably in the upward direction so that any gas bubbles that are formed are readily discharged from the cell's internal compartments and flushed out of the cell. Anolyte flows into the anolyte compartment 608 at inlet 612A and out at outlet 612B. From there the anolyte now enriched in hydrogen peroxide flows to a sterilization solution storage tank 620. Optionally the storage tank 620 may be omitted if the hydrogen peroxide is used immediately. The anolyte then flows to a treatment volume 624 (such as a sterilization tank) where treatment occurs. Soiled or contaminated devices or materials 626 are placed in the treatment volume (e.g. medical tools, devices) for treatment. The contaminated devices or materials are preferably pre-rinsed or precleaned to aid in the decontamination process. The sterilized or decontaminated devices or materials 628 are removed and remaining treatment materials removed as waste purge. If desired, electrolyte from the treatment volume 624 may be returned to the storage tank 620. Electrolyte from storage tank 620 is circulated back to the inlet 614A of the catholyte compartment 610 with pump 650 or entirely fresh make up electrolyte used. The entering catholyte (at inlet 614A augmented by makeup and/or return electrolyte flows through the catholyte compartment 610 and interacts with the cathode 604. The catholyte 604 then exits at outlet 614B and flows to a gas/liquid separator 630. Hydrogen and other gases produced in the cell can be removed at this point in the process. The outlet of the gas/liquid separator allows electrolyte to flow to enter the anolyte compartment 608 at inlet 612A. Make up water or additional electrolyte may enter at inlet 640 through an optional valve 642. Additional electrolyte chemicals or may be added at tank 620 via inlet 621. Sensors such as peroxide sensors can be inserted at inlet 620 also.

Another advantage of using pH buffers for the electrolyte is that the electrolyte does not become a hazard by being too acidic or basic. For the above carbonate example the pH is expected to range in the about 6 to about 10 ranges with most performance in the about 7 to about 9 range.

The Na₂CO₃ that is produced causes pH to rise in the catholyte and decrease in the anolyte. When the cell is operated with a divider membrane or porous frit, the pH of the bulk electrolyte will remain in a narrower range. Such mild pH values and ranges enable the disinfection capability to be adjusted over a range of aggressive (pH values>8) to milder (pH values<8). Note also that such mild pH conditions are preferred due to the mild corrosivity of NaHCO₃ and Na₂CO₃ solutions and their mixtures. Note also that sue to the H₂O₂ generated, that other spoilage preservatives normally required in other cleaning systems are not required in this case.

### Bias Voltage

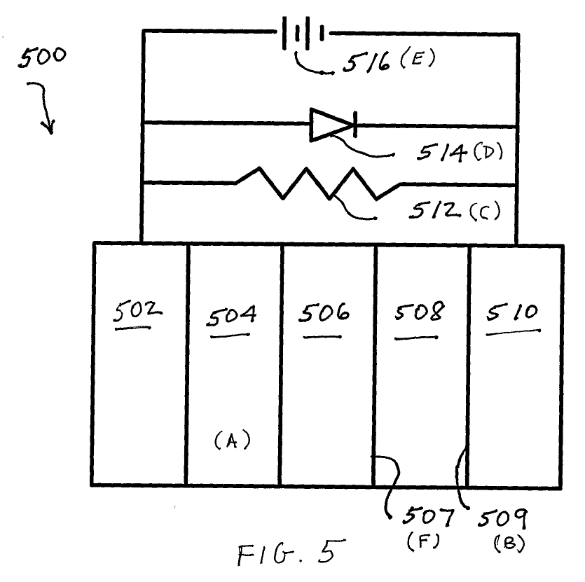

As shown in FIG. 4, the photolytic cell can optionally include a source for application of a bias voltage 449, or electrical load such as a motor or battery charger 452, which can be located in series or parallel to the conductor linking the cathode to the photocatalyst. This bias voltage can be supplied externally or internally to the cell, or a combination of these. If supplied in parallel, then, as is known to those skilled in the art of electronic circuitry that the other circuit in parallel must not be simply a metal conductor. For example it can contain a resistor or other load (FIG. 5). Other than an external applied voltage source, other means to impart a bias voltage is to incorporate a N/P semiconductor junction at the interface of the photocatalyst film and current collector film (see FIG. 5). An electrical current formed from the photocatalyst provides electrons to flow from the anode 36 to the cathode 38. The initial bias voltage supplied as described directs the current flow direction by initiating the removal of electrons formed during the conversion of water to H₂O₂ and prevents the electrons from reacting with the exciton or H₂O₂ to reform water. The bias voltage also allows more H₂O₂ to be produced as the removal of the electrons minimizes the reformation of water. Additional external electrical contacts can monitor or apply a particular voltage to the photolytic cell.

Once the direction of electron flow is initiated from photocatalyst to cathode, then, in the case of FIG. 5, the application of applied bias voltage is preferably minimized, or most preferably discontinued altogether, as the cathodic reaction is sufficiently favorable to polarize the cell. Note that the P/N junction provides a continuous passive bias voltage and so does not need to be turned on or off once in place and so is a most preferred case.

Referring again to FIG. 5, this figure is an illustration of optional bias voltage to photoelectron chemical cell (undivided cell illustrated). Also applicable to divided cell.) Note that Component 512 (C below) is always optional, while the requirements for one or more of components 502 (A), 514 (D), and 516 (E) depends on how facile the electrochemical reaction is at the interface 509 (B) and the photocatalyst reaction is at interface 507 (F).

A. P/N junction 512 (or its equivalent) that spontaneously forms a bias voltage that attracts electrons from photocatalyst 506, thereby shunting them to the current collector502.

B. Electrochemical reaction at cathode/electrolyte interface 509. The potential difference across this interface (voltage) and catholyte composition control which cathodic reactions can occur thermodynamically. The composition of the cathode 510 temperature, and of the electrolyte 508 can control the electrochemical reduction reaction rates.

C. Represents optional load 512 on the circuit that is available to achieve photolytically powered electrical energy provided cell potential generated is sufficient to do so. Note that cell voltage can be increased over that of a single cell by configuration in series, for example as a DC amplifier already well known to those skilled in the art.

D. Illustration of a diode 514 located in the anode-to-cathode electrical connection insures that electrons flowing from the current collector to the cathode do not return to the anolyte compartment where reduction of H₂O₂ product back to H₂O can occur.

E. Optionally, applied external bias voltage 516 derived from a battery or DC power supply powered by another power source such as an AC power outlet, a photovoltaic cell, battery, fuel cell, or the like.

H₂O₂ production rate is controlled y lamp or photon flux that is in turn controlled by the lamp power supply. Load 512 (C) in FIG. 5 represents optional load on the circuit that is available to achieve photolytically powered electrical energy provided cell potential generated is sufficient to do so. Note that cell voltage can be increased over that of a single cell by configuration in series, for example as a DC amplifier already well known to those skilled in the art. For a particular light flux and cell design, increasing the resistance of C, lowers the number of electrons (voltage) flowing from the anode to the cathode, thereby lowering the overall production of dissolved hydrogen peroxide. Decreasing the resistance of C, increases the flow of electrons from the anode to the cathode, thereby increasing the amount of hydrogen peroxide produced. In this manner the production rate of hydrogen peroxide can be controlled simply and automatically by conventional means known to those skilled in the art of electronic process controls.

### Optimal Gas Sorption Device 24

The cathodic electrochemistry should be selected to promote fast and effective H₂O₂ production in the anodic compartment by rapidly consuming electrons at sufficiently low voltages. A summary of such candidates, both organic and inorganic is provided in the Table 1 in PCT/US06/34004 filed Aug. 31, 2006, for Power Device and Oxygen Generator. In this manner gaseous products can be avoided by suitable selection of half-cell reactions for the cathodic compartment.

Hydrogen gas generation is also an option for the cathodic reaction and can readily be collected or, preferred for H₂O₂ generation, exhausted safely. As only small amounts of H₂ are expected for most H₂O₂ sterilization needs, and since the lightness of the H₂ allows easy venting this a preferred practice mode for the invention.

Hydrogen gas produced in the cathode will accumulate unless vented. H₂, being an extremely small molecule, readily diffuses through most non-metallic materials, especially plastics, ceramics, etc. The venting of H₂ can be controlled by selecting porous materials of construction that allow diffusion. No particular membranes, vessels, pumps, filters, one way valves, etc. are required to diffuse our the H₂. Hydrogen peroxide is not appreciably volatile and so will not be lost in this process. The other electrolyte compounds are also selected to have low volatility, such as water and inorganic salts.

### Light Supply 320

The light supply is used in the photolytic cell to provide the photon energy necessary to activate the catalyst converting water into hydrogen peroxide. The light source can be from any known light source including, but not limited to, sunlight, UV light, laser light, incandescent light, etc., depending on the activation requirement for the light activated catalyst used. UVA light and short wavelength visible light is most preferred in the range of about 350 to about 400 nm. If the design of the cell is to include illumination through the anolyte, then light wavelengths shorter than a325 nm are least preferred since homolytic dissociation of H₂O₂ into two OH free radicals occurs.

Though broad spectrum illumination is effective in all cases, a particular wavelength range of light will be more efficient depending upon the catalyst used. When tungstate (WO₃) is used as a light activated catalyst, visible light is the most efficient to activate WO₃. When TiO₂ or ZnO is used as a light activated catalyst, the light source used is most optimal in the UVA range.

Preferably, the light source used in the photolytic hydrogen peroxide generator is light in the range of about 350 to about 400 nm. Doped metal oxide photocatalysts with or without dye sensitized metal oxide photocatalysts, extends this range to about 450 nm.

Laser illumination is far more selective than broadband illumination. The wavelength of laser light can be manipulated in order to attain a higher efficiency in exciting the light activated catalyst and forming H₂O₂. Also, laser light allows the photolytic hydrogen peroxide generator to dissipate less overall heat. The laser light can be directed in a small wavelength range to energize the light activated catalyst and avoid contact or irradiation with other components of the photolytic hydrogen peroxide generator. A particularly preferred laser light that can be used to activate TiO₂ is an argon laser at 364 nm (400 mwatts/cm²), which has a total power of about 2 watts, although other UV sources, including an Hg arc lamp at 365 nm line, and tunable dye lasers are also available.

The optics for illumination are also important. It is preferred that the light from the light source be evenly spread within the photocatalyst film. The even spreading of the light from the light source allows for maximal excitation of the catalyst in order to convert more water into either activated oxygen or hydrogen peroxide. Along these lines, light from the light source can enter the photolytic cell through the transparent window from many positions. Light from the light source can enter directly through the transparent window and come into contact with the catalyst. Alternatively, light can enter the transparent window from a side, edge, back, bottom, through or corner position and move through the transparent window by a wave guide to provide photon energy and excite the light activated catalyst. Side entry of light into the transparent window of the photolytic cell occurs at about at least a 68° angle of incidence. Preferably, side entry of light into the transparent window occurs at an incident angle of from about 70° to about 80°. When the electrolyte is transparent to at least a portion of the UV-VIS spectrum (190-750 nm) then illumination through the electrolyte is also effective.

### Pump

A pump drives aqueous solution through the photolytic hydrogen peroxide generator. The pump draws the aqueous solution from a treatment volume and moves solution through the photolytic hydrogen peroxide generator. Preferably, the photolytic hydrogen peroxide generator only requires a pump to draw solution from the treatment volume, as the flow produced by the pump drawing solution from the treatment volume also moves the solution through the photolytic cell for activated oxygen formation and then back to the treatment volume. Although multiple pumps can be used, most preferred is that this single pump also moves the fluid through the catholyte compartment.

### Sensors Monitoring Reaction Chemistry

The photolytic hydrogen peroxide generator can include one or more sensors that monitor the different chemical reactions occurring within the photolytic cell. The sensors can be used to measure for redox potential, spectral properties, pH, to measure the sterilization strength of the product H₂O₂ and H₂O₂ production efficiency. Various sensors and sensor systems can be used including visual observations of color changes of redox indicator dyes or gas bubble formation, closed electrical current measurements and pH measurements, and dissolved oxygen probe analysis. Gas chromatography assays can also be performed. The catholyte can be similarly monitored. A dissolved oxygen probe can be used to test and monitor O₂ generation, as dissolved oxygen, in real time. Also, the photolytic hydrogen peroxide generator can incorporate one or more portals to insert a dissolved oxygen probe, CO₂ probe, pH monitor, electrical current flow, etc. in different locations if necessary. The photolytic hydrogen peroxide generator can also incorporate separate sampling chambers to trap gas bubbles for testing. These sampling chambers could also incorporate a device, such as a septum for a hypodermic needle for instance, to obtain a sample for further testing. One skilled in the art would recognize numerous sensors could be used for monitoring the reaction chemistries occurring within the photolytic cell.

The photolytic hydrogen peroxide generator and photolytic cell can also include one or more process regulator devices that respond to the readings provided by the sensors. The process regulator devices increase or decrease the amount of dissolved oxygen or CO₂ output, lower toxin levels, etc., depending on the requirements of the treatment volume or of the photolytic cell. It is within the purview of one utilizing the photolytic hydrogen peroxide generator to determine what process regulator devices are required. In addition, filtration of the electrolyte to about 0.02-10 micron is expected to help extend bath life, as will formulation with amino phosphonate metal ion chelators, stannic colloids, pyrophosphate chelators and the like.

All of the seals in the photolytic hydrogen peroxide generator are typically made of an inert material that is corrosion resistant and properly seals aqueous hydrogen peroxide solution flowing through the photolytic hydrogen peroxide generator from contamination. The seals of the photolytic hydrogen generator should also be formed of a material that does not interact with the activated oxygen, electrolyte, or hydrogen peroxide. Preferably, the seals are formed of Teflon, aluminum metal, PVC, PP, Viton® or silicone-based materials.

Optionally, laminar flow exists within the photolytic hydrogen peroxide generator. Internal mixing is accomplished by using flow dispensing designs common in current commercial cells, such as electrodialysis, electrodeionization, nanofiltration, microfiltration, RO, etc. Commercially available cells accommodate electrodes, membranes, and thin liquid chambers with flow distributors, and provide good seals and corrosion resistance. The cells are available in full commercial lab scale units for process development work. Particularly preferred commercial cells are the FMOL-LC device from ICI Chemicals and Polymers, Electrochemical Technology, Cheshire, UK, ElectroSyn, Inc., and the like.

### Multiple Photolytic Cells

Preferably, the photolytic hydrogen peroxide generator uses a plurality of photolytic cells in a “stacked” formation. The plurality of photolytic cells receive aqueous solution flow in parallel or serial configuration from the treatment volume and are exposed to photo-activation via a directed light source described above. The stacking of a plurality of photolytic cells allows for a large overall surface area for aqueous solution to receive maximal concentrations of generates hydrogen peroxide and/or to develop higher external voltages and/or DC current for powering external loads and/or cathode chemical reactions. Also, stacking a plurality of photolytic cells allows the overall photolytic hydrogen peroxide generator to achieve a smaller size than free standing individual cells, thereby allowing the photolytic hydrogen peroxide generator to be miniaturized.

### Photolytic Cell has Broader Applications

The photolytic cell as described may be used for photochemical processes beyond the preferred embodiments described above. These include but are not limited to production of products at the cathode, hydrogen peroxide production in general, point-of-use bleaching applications for applications such as metal surface finishing, wood pulp bleaching, DNA finger printing, water purification and the like.

### Preparation of Photocatalyst Preparation on Silica Glass or Quartz Slide 330

A glass surface was degreased by swirling in toluene or MEK or other degreasing solvent. The slide was flash dried in air for less than about 1 minute. The slide was then soaked in warm Micro® cleaning solution for about 2 minutes. The slide was then rinsed thoroughly with 18MΩ deionized water. The slide was immediately thereafter soaked in a water bath for about 2 minutes. The slide was rinsed thoroughly using a steady stream of deionized water and drained but not allowed to dry. With caution, the slide was submerged in a solution of concentrated sulfuric acid and was allowed to stand for 2 minutes. A polypropylene plastic hemostat was used to hold the glass plate when it is inserted/withdrawn from the sulfuric acid. The plate was withdrawn, allowed to drain, and rinsed thoroughly with deionized water. The plate was then soaked in a water bath for about 2 minutes. A water break test was then performed on the plate to verify clean lines, testing positive for being clean. Using a plastic (Nalgene®) beaker with cover, the slide was dipped for 2 minutes in a solution of 0.1% HF and 1N HCl. The surface of the glass now contained Si—OH linking groups. These plates were kept wet, and stored in pure 5% HNO₃.

### Catalyst Layer 332 Preparation by the Sol-Gel Method

About 1.0 g of TiO₂ (anatase) was added to a plastic (Nalgene®) beaker with a cover watch glass, and a magnetic stir bar. In a hood, 80 mL of 0.1% HF and 1N HCl was added to the TiO₂. A magnetic stirrer mixed the contents of the beaker until the solids were well suspended. The beaker was mixed for 60 seconds and process proceeded immediately to the next step of dividing the slurry between two 50 mL capped centrifuge tubes. The tubes were centrifuged for at least 5-10 minutes. The supernatant was discarded. Each tube was rinsed 3 times with 40 mL portions of water. The tube was capped, vortexed thoroughly, centrifuged, decanted, and the steps were repeated. Each tube was rinsed 3 times with 40 mL portions of isopropanol (iPrOH). Preferably, one or more inorganic silane and/or titanate-coupling agents can be added to the last alcohol rinse to facilitate agglomeration and adhesion in the final coating. The aggressive oxidizing environment of the UV/TiO₂ during use may rapidly degrade organic-based coupling agents and so inorganic couplings are most preferred.

### Application of the Photocatalyst to the Glass Plate

The pretreated TiO₂ anatase particles were stirred to re-suspend the solids from one of the tubes in the above preparation in a jar containing isopropanol sufficiently deep to cover the glass microscope slide. Magnetic stirring was initiated to keep the particles suspended. The amount of particles, emerging time, and emerging temperature used is an adjustable parameter in determining the thickness of the final coating produced.

A sufficient amount of Ti(iOPr)₄ (TTIP) was added to yield a 0.2 vol % solution (e.g., by adding 160 uL TTIP per 80.0 mL isopropanol). Using a plastic hemostat to hold the slide, the treated glass plate was rinsed thoroughly with water and was again tested under the water break test. The slide surface was rinsed thoroughly with isopropanol. The slide was soaked for 2 minutes in isopropanol and rinsed again with isopropanol. The slide was immediately hung in the TTIP/isopropanol solution and stirred. The vessel was covered to minimize pickup of moisture from the air, and allowed to react for about 120 seconds. During this time, the TTIP reacted with the Si—OH groups on the surface of the glass slide to form O—Si—O—Ti-iOPr linkages, although the linkages may not have formed completely until the heating step below. The slide was removed very slowly (e.g. 1 cm/min) using the hemostat manual or automated retrieval methods and was laid flat on an inverted small relative area polyethylene support in a vacuum desiccator to dry for a few minutes. The standing time in the room air (humidity level and contact time) was an adjustable parameter since water vapor diffuses to the surface of the slide causing hydrolysis reactions (the “sol” in sol-gel), i.e.,

Ti(iOPr)₄+2H₂O→TiO(iOPr)₂+2iPrOH

TiO(iOPr)₂+2H₂O→TiO₂+2iPrOH

Excess water must be avoided so that the silanol groups on the surface of the slide may also react in competition with H₂O present, i.e.,

glass surface−Si—OH+Ti(iOPr)₄→Si—O—Ti(iOPr)₃+iPrOH

Similar reactions couple the TiO₂ anatase particles to the surface of the glass or quartz and to each other (the “gel” in sol-gel),

TiO₂(anatase)-Ti—OH+Ti(iOPr)₄→TiO₂(anatase)-Ti—O—Ti(iOPr)₃+iPrOH

It is noted, however, that thoroughly desiccated (water-free) surfaces are also not useful since then dehydration of surface Si—OH and Ti—OH groups to SiO₂ and TiO₂ occurs, which would remove the hydrogen ion needed to produce the iPrOH product at low energy. The time spent at this room temperature condition can be adjusted since the coating slowly reacts during this time. Hence the time-temperature profile is a film formation control factor.

While still lying flat, the slide is oven-dried at 80-90° C. for 20 minutes to finish the cure. The time, temperature and heating rate (° C./min) parameters are adjustable. Heating too fast can blow out solvent, causing massive disruption and porosity of the film due to out gassing, while heating too high a temperature can cause too much condensation resulting in shrinkage, leading to pulling away of the film and cracking. Porosity is expected to be important so that water can penetrate and hydrogen peroxide can leave the reaction zone. Such time-temperature relationships are well understood to those skilled on the art of sol-gel film preparations.

In order to obtain slides having a thicker TiO₂ coating, the above steps are repeated one or more times and/or for extended times and at greater temperatures. For these cases where illumination through the electrolyte is planned metal conducting opaque substrates can be used, including aluminum, copper, silver, gold, platinum, and the like.

The slide (plate) was heated to 250° C. for two hours to fully cure and set the coatings. This temperature was needed to convert the amorphous TiO₂ formed from the TTIP into anatase. (Ind. Eng. Chem. Res. 1999 38(9), 3381). Alternatively, a slide can be pretreated as above except heat the coating to 350° C. at the rate of 3° C./min and hold at this temperature for 2 hr. (Miller, et al. Environ. Sci. Technol, 1999, 33, 2070). Another alternative is to prepare the sol-gel solution in place of the anatase/TTIP slurry. (Colloid C in Aguado, M. A., et al., Solar Energy Materials, Sol. Cells, 1993, 28, 345). The slide was then removed and allowed to cool to room temperature.

The coating adhesion of the TiO₂ anatase to the glass slide was tested by abrasion with a rubber policeman, tape test, etc. Also, the coating adhesion was tested for other properties including thickness, tendency to crumble/flake off, visual appearance, etc.

The experiments were repeated as needed to improve adhesion and other properties. An additional step of a 400° C. treatment for one hour can used to set TiO₂ (anatase) particles onto a quartz sand slide (Haarstrick, et. al. 1996). Vacuum sputtering TiO₂ film formation techniques are also effective in forming such photocatalyst films, especially onto inner layers of conductor film of ITO, Ti, Au or Sn, and the like.

### TiO₂ Coating Photochemistry Testing

Two TiO₂ coating photochemistry testing procedures were conducted, the first to determine whether electrons were generated and the second to determine whether activated oxygen was generated. First, the TiO₂ was tested by a negative charge/electron generation test. Methyl viologen (MV²⁺) blue color (MV⁺) was applied onto the anatase coating and was subjected to UV argon laser light. A rapid appearance of dark blue color was observed to form, thereby qualitatively validating electron formation. The MV⁺ blue color was not permanent since MV⁺ is a free radical/charge transfer complex, which easily releases e⁻ and returns to colorless ground state. Dried coating inhibited the performance of coating (dried minerals block surface sites), but was easily cleaned.

A second test conducted on the TiO₂ coating layer was the activated oxygen generation test. Methylene blue was used on the TiO₂ coating to determine the presence of activated oxygen. The methylene blue color was rapidly destroyed at the point of the laser light in the presence of anatase coating, validating activated oxygen formation, since oxidized oxygen reacts with methylene blue to discharge its color by reducing methylene blue's aromaticity.

### Light Source

The light source used above was an argon laser at 364 nm line (400 mwatts/cm²) available (tunable to lower powers). The argon laser used has a total power of 2 watts. Alternatively, a number of UV sources were screened, including Hg arc lamps filtered to using a 365 nm line.

### Anode Conductor Layer 336

The anode conductor or current collector layer was formed by placing a very thin film of uniform metallic conductor having a thickness of less than about 0.2 um using e-beam vapor deposition onto a transparent window. The thin film was formed of Ti metal. Conductor line spacing, width and thickness optimization may be used for controlling the anode conductor layer thickness and chemical composition and physical structure to prevent excessive attenuation while providing sufficiently close and intimately contacting conductive areas to sweep photolytically generated exciton electrons away from TiO₂ layer to prevent their recombination with H₂O₂.

### Hydrogen Peroxide Generating Catalyst Layer 34

A hydrogen peroxide generating catalyst layer is formed from ZnO particles coated onto the surface of the TiO₂ (anatase) layer. The ZnO particles are applied from a An(OH)₂ slurry with or without the anatase/Ti(iPrO)4 mixture. A significant amount of the surface of the TiO₂ (anatase) layer is coated (˜⅓) by the ZnO. Adding the Zn dropwise and allowing it to evaporate is effective. The ZnO is added to increase % surface area covered by ZnO particles and to make the ZnO more adherent using the Ti(iOPr)₄ binder. Formation of ZnO then films using carefully controlled version of the glycine/zinc nitrate method was also effective.

### Flow Through Cell

In one example, the flow through cell was designed with fluid inlets and outlets on the same side. Silicone gaskets and spacers, acrylic external housing and stainless steal tubing connectors were used in forming the flow through cell. In the flow through cell, the anode was the continuous Ti plate and the cathode was a platinum foil strip.

### Electrical Connection of Flow Through Cell

The electrical connection of the flow through cell was wired as a shorted circuit with a current meter and externally applied bias voltage inline. The electrical connection of the flow through cell could also be formed by applying bias voltage added as described above. The electrical connection of the flow through cell could also be formed by placing a resistor and a current meter inline with a voltage reading across a resistor.

### Divided Cell

A divided cell was designed with both sets of fluid inlets and outlets on the same side with the through-anode, through-acrylic housing and silicone spacer internal flow paths and on the side opposite the glass slide. The divided cell was further designed to include silicone gaskets or spacers, acrylic external housing, NAFION™ cation exchange membrane, and 3163L stainless steel tubing connectors.

### Activated Oxygen Testing

A Locke's Ringer saline pH buffer test solution was prepared with 150 ppm redox dye (methyl viologen, MV²⁺). Also, a 10 uM solution of methylene blue was prepared in the Locke's Ringer solution. (Matthews, R. W., J. Chem. Soc., Faraday Trans. 1, 1989 85(6), 1291.) The molar absorbtivity for methylene blue at 660 nm is 66,700±350 cm⁻¹M¹. The coated test slide was assembled with an attached UV lamp/laser. The Locke's Ringer solution was then added to the coated test slide in and assembled floe-through cell via a circulating pump. After steady conditions were attained, the coating was illuminated directly/indirectly with UV light. The saline solution was monitored for appearance of blue color (MV²⁺(colorless)+e−→MV⁺(blue)) and dissolved oxygen. Gas samples were sampled for GC assay (for CO₂ and O₂ when the system is operated sealed against entry by air).

### Results

The hydrogen peroxide generator was tested in order to determine whether the chemical formulations occurred as predicted. The testing was conducted using Locke's Ringer solution, which is a pH buffered saline solution. The qualitative results of the testing are as follows:

1. Highly efficient U.V. light absorption by thin films of TiO₂ (anatase) to impart energy into the anatase matrix was visually apparent in that the UV light is substantially absorbed. Attenuation by any metal conducting film present was measured and corrected separately.

2. Generation of activated oxygen (AO) at the anatase surface using the energy from the UV light was evidenced by methylene blue dye disappearance at the surface of the anatase film opposite the side irradiated by the UV laser.

3. Generation of free electrons (e⁻) at the anatase surface, when the current collector is not electrolytically connected to the cathode and no bias voltage is applied, using the energy from the UV light was evidenced by methyl viologen blue dye color appearance at the surface of the anatase metal oxide photocatalyst film applied on the side of the glass or quartz plate on opposite the side irradiated and only at the location of irradiation.

4. Transport of the free electrons (e⁻) generated above to a conductive Ti anode surface, which were then swept away so that the free electrons do not recombine with the activated oxygen also produced above was evidenced by electrical current in the photocatalyst semiconductor film, through a metallic current collector, wire and amp meter. The electrical current was found to flow only when the laser was on and the electrical current never flowed when the laser was off. The effect was observed through numerous off/on cycles, and the electrical current measured was proportional to the laser intensity up to a saturation point.

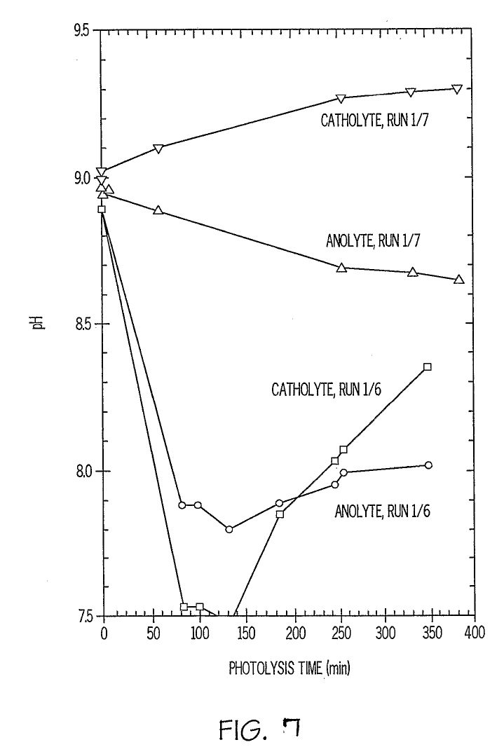

5. The release of hydrogen ions (H⁺) and pH drop was found for the anodic compartment in a continuously circulated and irradiated enclosed cell. The opposite pH change was found for the cathodic compartment, which was consistent with the pH effect expected when water is separated into activated oxygen and hydrogen ions at the metal oxide catalyst surface. FIG. 7 shows a plot of the pH profile of the anolyte and catholyte during photolysis using the photolytic cell. The opposite trends in the plot are as predicted based on the proposed chemistry, decrease in pH in the anolyte and a pH increase in the catholyte. The lower initial pH in the catholyte in Run ⅙ reflects a startup condition with a slightly lower pH. Run 1/7 used a pre-equilibrated photolytic cell to remove any inconsistent readings during start up conditions.

6. The conversion of HCO₃⁻ ions from the electrolyte, i.e., Locke's Ringer solution, into CO₂, can be observed by the formation of more H₂O. H₂O is the expected product to be formed along with CO₂ during the bicarbonate ion conversion to carbonic acid and ultimate conversion to H₂O and CO₂ using the H⁺ ions released during the formation of activated oxygen. CO₂ production was measured by gas chromatography (GC) analysis of off-gases, or calculated from pH changes. The CO₂ level found by GC analysis was significantly greater than atmospheric level, further indicating the formation of CO₂.

7. The generation of alkalinity at the cathode and related pH change indicated that the free electrons produced during the reaction of water into activated oxygen were conducted away from the anode and consumed in a non-O₂ reducing manner, i.e., by reduction of water to hydroxide ion and H₂ gas at the catalyst.

Broad embodiments of the invention provide for a photolytic hydrogen peroxide generator include
a photolytic cell having a light activated catalyst, the light activated catalyst converts water to hydrogen peroxide;
an optional porous sealant layer disposed on the light activated catalyst and separating the light activated catalyst from a solution circulating thought the photolytic cell;
a light supply providing light to the photolytic cell and activating the light activated catalyst;
a pump circulating a solution through the photolytic cell;
an inlet, transporting the solution into the photolytic cell; and
an outlet transporting the solution out of the photolytic cell.
Another broad embodiment provides for a photolytic hydrogen peroxide generator including a photolytic cell having a light activated catalyst, the light activated catalyst converts water to hydrogen peroxide, and wherein the light activated catalyst comprises two layers, a first layer for capture of photons and charge separation and a second layer adjacent to the first layer for hydrogen peroxide production;
an optional porous sealant layer disposed on the second light activated catalyst layer and separating the second light activated catalyst from a solution circulating thought the photolytic cell;
a light supply providing light to the photolytic cell and activating the light activated catalyst;
a pump circulating a solution through the photolytic cell;
an inlet, transporting the solution into the photolytic cell; and
an outlet transporting the solution out of the photolytic cell.
An additional embodiment provides for a photolytic cell including a transparent window;
an anode conductor layer adjacent to the transparent window;
a light-activated catalyst disposed upon the anode conductor layer, wherein the light activated catalyst produces hydrogen peroxide;
an anolyte adjacent to and bordering the catalyst;
a divider bordering the anolyte to form a first volume,
a catholyte bordering the divider, and
a cathode bordering the catholyte to form a second volume.
A further embodiment provides for a method for delivering activated oxygen to a solution comprising:
moving solution into a photolytic cell;
converting water into hydrogen peroxide by a light-activated catalyst in the photolytic cell;
binding the hydrogen peroxide to the solution; and
moving the solution out of the photolytic cell.
A yet further embodiment provides for
a method for providing hydrogen peroxide to a treatment volume including moving an electrolyte into a photolytic cell;
converting water to hydrogen peroxide in the photolytic cell;
forming hydrogen in the photolytic cell;
removing hydrogen formed in the photolytic cell and electrolyte; and
moving electrolyte out of the photolytic cell.
   A yet further embodiment provides for an apparatus for producing hydrogen peroxide including
a. a waveguide layer for conducting light;
b. a first conductor layer adjacent to the waveguide;
c. an active layer on the other side of the conductor and adjacent to the conductor;
d. a first volume having an inlet and an outlet bounded at least in part by the active layer;
e. a divider bounding at least a portion of the first volume; and
f. a second volume on the opposite side of the divider from the first volume having an inlet and an outlet that is bounded at least in part by the divider

The invention has been described with reference to the preferred embodiments. Obviously, modifications and alterations will occur to others upon a reading and understanding the preceding detailed description. Particularly, it is clear to one having ordinary skill in the art that the photolytic cell can be modified and used in numerous other reactions and reaction systems. Furthermore, one skilled in the art would appreciate based upon the preceding detailed description that the photolytic generator can be used in forming chemical reactions in solutions other than aqueous solutions. It is intended that the invention be construed as including all such modifications and alterations in so far as they come within the scope of the appended claims or the equivalents thereof.

Having thus described the preferred embodiment, the invention is now claimed to be:

1. A photolytic hydrogen peroxide generator comprising:
a photolytic cell having a light activated catalyst, the light activated catalyst converts water to hydrogen peroxide;
an optional porous sealant layer disposed on the light activated catalyst and separating the light activated catalyst from a solution circulating thought the photolytic cell;
a light supply providing light to the photolytic cell and activating the light activated catalyst;
a pump circulating a solution through the photolytic cell;
an inlet, transporting the solution into the photolytic cell; and
an outlet transporting the solution out of the photolytic cell.

2. A photolytic hydrogen peroxide generator comprising:
a photolytic cell having a light activated catalyst, the light activated catalyst converts water to hydrogen peroxide, and wherein the light activated catalyst comprises two layers, a first layer for capture of photons and charge separation and a second layer adjacent to the first layer for hydrogen peroxide production;
an optional porous sealant layer disposed on the second light activated catalyst layer and separating the second light activated catalyst from a solution circulating thought the photolytic cell;
a light supply providing light to the photolytic cell and activating the light activated catalyst;
a pump circulating a solution through the photolytic cell;
an inlet, transporting the solution into the photolytic cell; and
an outlet transporting the solution out of the photolytic cell.

3. The photolytic hydrogen peroxide generator of claim 2, wherein the photolytic hydrogen peroxide generator further comprises a gas separation device connected to the photolytic cell.

4. The photolytic hydrogen peroxide generator of claim 3, wherein the photolytic cell releases gas into the gas sorption device.

5. The photolytic hydrogen peroxide generator of claim 2, wherein the photolytic hydrogen peroxide generator further comprises a sensor monitoring reaction chemistry in the photolytic cell.

6. The photolytic hydrogen peroxide generator of claim 4, wherein the hydrogen peroxide generator further comprises a processor regulating the photolytic cell in response to the sensor.

7. The photolytic hydrogen peroxide generator of claim 1, wherein the solution is an aqueous electrolyte.

8. The photolytic hydrogen peroxide generator of claim 2, wherein the photolytic cell converts water to dissolved activated oxygen.

9. The photolytic hydrogen peroxide generator of claim 8, wherein the dissolved activated oxygen converts to hydrogen peroxide.

10. A photolytic cell comprising:
a transparent window;
an anode conductor layer adjacent to the transparent window;
a light-activated catalyst disposed upon the anode conductor layer, wherein the light activated catalyst produces hydrogen peroxide;
an anolyte adjacent to and bordering the catalyst;
a divider bordering the anolyte to form a first volume,
a catholyte bordering the divider, and
a cathode bordering the catholyte to form a second volume.

11. The photolytic cell of claim 10, wherein the light-activated catalyst is a metal oxide catalyst.

12. The photolytic cell of claim 10, wherein the cell further comprises a second catalyst disposed on the light-activated catalyst.

13. The photolytic cell of claim 10, wherein the photolytic cell converts water into activated oxygen.

14. The photolytic cell of claim 10, wherein the light-activated catalyst converts water into activated oxygen.

15. The photolytic cell of claim 10, wherein electrons flow from the anode to the cathode.

16. A method for delivering activated oxygen to a solution comprising:
moving solution into a photolytic cell;
converting water into hydrogen peroxide by a light-activated catalyst in the photolytic cell;
binding the hydrogen peroxide to the solution; and
moving the solution out of the photolytic cell.

17. The method of claim 16, wherein the solution is an electrolyte.

18. The method of claim 16, further comprising removing gas from the solution in the photolytic cell.

19. A method for providing hydrogen peroxide to a treatment volume comprising:
moving an electrolyte into a photolytic cell;
converting water to hydrogen peroxide in the photolytic cell;
forming hydrogen in the photolytic cell; and
removing hydrogen formed in the photolytic cell and electrolyte; and
moving electrolyte out of the photolytic cell.

20. The method of claim 19, further comprising removing reacted hydrogen peroxide product from a treatment volume.

21. The method of claim 19, further comprising returning electrolyte containing hydrogen peroxide to a treatment volume.

22. A method for disinfecting a surface or volume comprising:
a. producing hydrogen peroxide by photolytic generation from water containing a buffer with light using a semiconductor material;
b. applying the produced hydrogen peroxide to a surface or volume.

23. The method according to claim 22, wherein the hydrogen peroxide is produced in the presence of a stabilizer.

24. The method according to claim 22, wherein the hydrogen peroxide is produced in the presence of a buffer.

25. An apparatus for producing hydrogen peroxide comprising:
a. a waveguide layer for conducting light;
b. a first conductor layer adjacent to the waveguide;
c. an active layer on the other side of the conductor and adjacent to the conductor;
d. a first volume having an inlet and an outlet bounded at least in part by the active layer;
e. a divider bounding at least a portion of the first volume;
f. a second volume on the opposite side of the divider from the first volume having an inlet and an outlet that is bounded at least in part by the divider
g. a second conductor layer bounding at least a portion of the second volume, wherein the second conductor does not come in contact with the divider; and
g. a disinfecting region having an inlet that is operationally connected to the outlet of the first volume.
